(19) United States
(12) Patent Application Publication
(10) Pub. No.: US 2007/0092444 A1
(43) Pub. Date: Apr. 26, 2007

(54) Inhibition of inward sodium currents in cancer

(75) Inventors: Dale J. Benos, Birmingham, AL (US); James K. Bubien, Pelham, AL (US); G. Yancey Gillespie, Birmingham, AL (US)

Correspondence Address: BRADLEY ARANT ROSE & WHITE, LLP;INTELLECTUAL PROPERTY DEPARTMENT-NWJ, 1819 FIFTH AVENUE NORTH, BIRMINGHAM, AL, 35203-2104, US

(73) Assignee: THE UAB RESEARCH FOUNDATION, Birmingham, AL (US)

(21) Appl. No.: 10/571,302

(22) PCT Filed: Sep. 13, 2004
(86) PCT No.: PCT/US04/29970

§ 371 Date: Aug. 17, 2006

Related U.S. Application Data
(60) Provisional application No. 60/502,034, filed on Sep. 11, 2003.

(51) Int. Cl. A61K 51/00; A61K 38/16; A61K 38/17

(52) U.S. Cl. 424/1.69; 514/12; 514/8

(57) ABSTRACT

Described is a constitutive inward Na⁺ currents found in a variety of human cancers. The constitutive inward Na⁺ current plays a role in increased cellular proliferation, cellular migration and volume regulation. The inward current is mediated, at least in part, by AISC-containing Na⁺ channels. In addition, an inhibitor of the inward current, the PcTX1 peptide, is described. Also provided are methods for screening compounds to inhibit the inward Na⁺ current, methods for screening for tumors expressing the inward Na⁺ current and methods for treating tumors expressing the inward Na⁺ current.

### FIELD OF THE DISCLOSURE

The present disclosure relates generally to inward constitutive Na⁺ currents and the Na⁺ channels mediating such currents, and to the identification, characterization and treatment of tumors expressing said Na⁺ currents.

### BACKGROUND

The ever-expanding Degenerin/ENaC (Deg/ENaC; ENaC=Epithelial Na Channel) superfamily contains over 60 proteins having a similar topology. As shown in FIG. 1, each family member has a short intracellularly located N— and C-termini, two predicted transmembrane spanning domains (M1 and M2), and a large extracellular loop (1,2). All family members are cation selective and blocked by the diuretic amiloride (1-3). Recently, another branch of this superfamily, the human BNaC (Brain Na Channel, also known as ASIC, Acid Sensing Ion Channel) family has been identified (4,5). The six members of this family so far identified in mammals are primarily expressed in the brain and in sensory organs.

Individual members of the ASIC family co-assemble to form heteromeric channels with differing properties, and are postulated to be involved in a wide variety of cellular responses ranging from nociception to mechanosensation (6,7). To date, six members of the BNaC/ASIC subfamily of the Deg/ENaC family have been cloned in mammals (5,39-42). Table 1 gives a summary of these channels and their pseudonyms. Each of these channels, except for ASIC2b, share the common characteristic of generating excitatory currents in response to acidic pH when studied in heterologous expression systems. ASIC2b, at least in its homomeric form, does not appear to respond to low pH. Although the subunit composition of these brain sodium channels in native tissues is unknown, evidence for heteromultimeric channel formation with distinctive functional characteristics has been obtained (6,43,44). A role in chemical pain sensation, especially that associated with increased acidification, has been proposed for these channels in sensory neurons (45,46).

Like the degenerins and ENaCs, ASICs are generally thought to form mechanically gated ion channels and to be involved in cell volume regulation (32,33). ASICs may also be involved in the small sodium influx that occurs in cells and thus contribute to the cell's resting potential. Alterations in membrane potential, either by activating or inhibiting these channels, may have deleterious effects on cell survival (34). Isolation of an inhibitor of these channels may be useful as a therapeutic agent as well as a diagnostic agent

### BREIF DESCRIPTION OF THE FIGURES

FIG. 1 shows the structure of the Deg/ENaC superfamily of amiloride-sensitive Na⁺ channels

FIGS. 2A-C show representative whole-cell patch clamp recordings. FIG. 2A shows the whole-cell patch clamp recordings from freshly isolated normal human astrocytes and GBM (WHO Grade IV), and primary cultures of different grades of glial tumors (astrocytomas); FIG. 2B shows the whole-cell patch clamp recordings in the presence of 100 uM amiloride; and FIG. 2C shows the amiloride-sensitive difference current.

FIGS. 3A and 3B show a summary of absolute outward (+40 mV; FIG. 3A) and inward (−60 mV; FIG. 3B) currents obtained from a variety of gliomas and normal cells in the absence and presence of 100 μM amiloride, using whole-cell patch clamp.

FIGS. 4A and B show summary I-V curves of freshly resected normal astrocytes (FIG. 4A) and GBM cells (FIG. 4B). Inward currents (−60 mV) were −7.5±1.2 pA (normal) and −43.8±14.5 pA (GBM). Outward currents (+40 mV) averaged 42.2±2.4 pA and 47.2±12.5 pA for normal and GBMs, respectively. FIGS. 4C and D show summary amiloride-sensitive (difference) currents of freshly resected normal astrocytes (FIG. 4C) and GBM cells (FIG. 4D).

FIGS. 5A-5C show representative whole-cell patch clamp recordings. FIG. 5A shows whole-cell patch clamp recordings from ZR-75-1 and SKMEL-2 cells; FIG. 5B shows the whole-cell patch clamp recordings in the presence of 100 uM amiloride; FIG. 5C shows the amiloride-sensitive difference current.

FIGS. 6A and B show RT-PCR detection of ASIC1 and ASIC2 in normal tissues, GBM tissues and cell culture samples. FIGS. 6A and B are the results of two separate experiments with partial overlap of tissues and cell lines tested. Primers for ASIC1 spanned bp 1091-1537 and bp 1109-1587+3′ UTR for ASIC2. N-normal control cells; G-freshly excised GBM; P-primary (1^{st} passage) GBM cells; astrocyte-primary (1^{st} passage) culture of normal human astrocytes.

FIGS. 7A-7C show representative whole-cell patch clamp recordings. FIG. 7A shows whole-cell patch clamp recordings from U87-MG, SK-MG, and D54-MG glioma cells in the basal state; FIG. 7B shows the whole-cell patch clamp recordings in the presence of 100 uM amiloride; FIG. 7C shows the amiloride-sensitive difference current. Amiloride (100 μW inhibited inward currents in all three cell types, regardless of the absence or presence of ASIC2 mRNA (FIG. 7D).

FIGS. 8A-C show acid-activated ASIC currents in Xenopus oocytes. ASIC 2 (FIG. 8A), ASIC1 (FIG. 8B) and the combination of ASIC2 and ASIC1 (FIG. 8C) were examined. Inward Na⁺ currents versus time were measured in voltage-clamped oocytes (−60 mV) in the absence and presence of 400 μM amiloride following activation by reduction of extracellular pH to 4.0 (solid bars). Each oocyte served as its own control. Each experiment was repeated three times with similar results.

FIG. 9 shows analysis of the interaction between ASIC1 and ASIC2 in proteoliposomes. In vitro transcription and translation of ASIC1 and ASIC2 were performed using either radioactive or non-radioactive methionine. Translated proteins were reconstituted into liposomes as per standard procedures known in the art. To test for co-precipitation, antibodies directed against non-labeled ASIC were used, and the presence of co-precipitated radioactively labeled ASIC was detected.

FIGS. 10A-C show co-immuno-precipitation of ASIC1, ASIC 2 and γ-hENaC from SK-MG cells. Whole cell lysate from SK-MG cells was immunoprecipitated using ASIC2 antibodies and probed on Western blots with antibodies against ASIC1 (FIG. 10A) ASIC2 (FIG. 10B) or γ-hENaC (FIG. 10C). Control immunoprecipitations were performed using IgG and probed on Western blots as indicated above.

FIGS. 11A-C show co-localization of syntaxin 1A and ASIC1 in SK-MG cells. All of the panels represent epifluorescent images. FIG. 11A: ASIC1 was stained using commercially available polyclonal anti-ASIC1 antibodies (Chemicon). FIG. 11B: Syntaxin 1A was stained using highly specific monoclonal antibodies (no cross reactivity between syntaxin 1A and syntaxin 1B). FIG. 11C: Double staining with anti-syntaxin 1A and anti-ASIC1 antibodies. Overlap is observed, as indicated by yellow.

FIGS. 12A-C show Co-localization of syntaxin 1A and γ-hENaC in SK-MG cells. All of the panels represent epifluorescent images. FIG. 12A: γ-hENaC was stained using a commercially available antibody (source). FIG. 12B: syntaxin 1A was stained using highly specific monoclonal antibodies (no cross reactivity between syntaxin 1A and syntaxin 1B). FIG. 12C: Double staining with anti-syntaxin 1A and anti-γ-hENaC antibodies. Overlap is observed, as indicated by yellow.

FIGS. 13A and B show expression and secretion of MT-SP1 in several glioma cell lines. FIG. 13A shows the presence of MT-SP1 in glioma cells lines SK-MG, SNB19, U87-MG and U251. MT-SP1 was not detected in normal astrocytes or in a Grade II astrocytoma. FIG. 13B shows gelatin zymography of proteases excreted from SK-MG cells. From left to right, lane 1 served as a control; lane 2, indicates treatment with 10 mM EDTA; lane 3 indicates treatment with 10 mM Aprotinin; and lane 4 indicates treatment with 10 mM of Galardin (Sigma-Aldrich), matrix metalloproteinase inhibitor.

FIGS. 14A and B show the effect of syntaxin 1A on ASIC1+ASIC2 (FIG. 14A) and ASIC1+ASIC2+γ-hENaC (FIG. 14B) in planar lipid bilayers. The holding potential was +100 mV and records were filtered at 200 Hz. Addition of syntaxin 1A was to the cis chamber; addition of syntaxin 1a to the trans side was without effect.

FIG. 15 shows the effect of syntaxin 1A on ASIC1, ASIC2, ASIC1+ASIC2 and ASIC1+ASIC2+γ-hENaC following expression in oocytes. Currents (Ip) were normalized to the values measured at −60 mV in the absence of syntaxin 1A. Currents were evoked by a step decrease in pHₒ to 4.0. Co-expression of syntaxin 1A with ASIC1+ASIC2+γ-hENaC resulted in significantly (P<0.01) lower mean currents.

FIGS. 16A-C show concentration dependent inhibition of cell proliferation of SK-MG (FIG. 16A), U373 (FIG. 16B), and U251 (FIG. 16C) glioma cells by amiloride, phenamil, and/or benzamil. Cells were plated in 96-well plates at 1000, 4000, and 2000 cells/well for SK-MG, U373, and U251 cells, respectively. Drug was added at specified concentration on day 3 after plating (at the beginning of log phase of growth).

FIG. 17 shows inhibition of Transwell migration of D54MG cells by benzamil. 5-8 μm polycarbonate Transwell filters were coated on the lower surface with or vitronectin (10 mg/ml in PBS). 100 ml of D54MG cells (400,000 cells/ml were added to the upper chamber), in the presence or absence of benzamil, and migration was allowed to proceed for 3 hours. Migration was determined according to standard procedures (120). N-amidino-3,5-diamino-pyrazinecarboxamide was used as a control. This pyrazine ring compound is an inactive analog of amiloride.

FIGS. 18A and B show the effect of PcTX1 (10 nM) and randomly scrambled control peptide (10 nM) on inward Na⁺ currents in a freshly resected GBM (FIG. 18A, upper panel), SK-MG cell (FIG. 18A, lower panel), or normal human astrocyte (FIG. 18B). As a control, a scrambled 40-mer peptide having the same amino acids as PcTX1 was used.

FIGS. 19A-19C show representative whole-cell patch clamp recordings. FIG. 19A shows whole-cell patch clamp recordings from ZR-75-1 and SKMEL-2 cells in the basal state; FIG. 19B shows the whole-cell patch clamp recordings in the presence of 100 uM PcTX1; FIG. 19C shows the PcTX1-sensitive difference current.

FIG. 20 shows the effect of PcTX1 (1 nM) and randomly scrambled control peptide (1 nM) on acid-induced ASIC currents in voltage-clamped Xenzopus oocytes. Membrane potential was held at −60 mV, and the pHₒ was step decreased to 4.0 for 10 s, and then returned to 7.4 for 30 s before repeating the sequence. Oocytes were superfused with PcTX1 solution (solid bars). PcTX1 only inhibited inward currents mediated by ASIC1a and not the inward currents mediated by ASIC2 or the combination of ASIC1 and ASIC2. The control scrambled peptide was without effect.

FIGS. 21 shows single channel recordings of the ASIC1 reconstituted into planar lipid bilayers in the absence (upper panel) and in the presence (lower panel) of the PcTX1. An expanded time scale is shown below each trace.

FIGS. 22A-B show the effect of PcTX1 on kinetic properties of the ASIC1 in planar lipid bilayers. The number of events used for construction of the closed and open time histograms shown were: 811 and 812 (FIG. 22A, in the absence of the PcTX1) and 989 and 988 (FIG. 22B, in the presence of 10 nM PcTX1).

FIGS. 23A and B show single channel records of ASIC-containing channel activity in cell attached (FIG. 23A) and outside-out patches (FIG. 23B) from U87-MG cells.

FIGS. 24A-D show the effect of PcTX1 or randomly scrambled control peptide on cell migration in U87-MG cells (FIG. 24A), D54-MG cells (FIG. 24B), primary GBM cultures (FIG. 24C) and primary human astrocytes (FIG. 24D) cells.

FIGS. 25 shows the time course of regulatory volume increase (RVI) in U87-MG cells following osmotic shrinkage with no peptide added (control) or in the presence of 80 nM PcTX1 or randomly scrambled control peptide was added. U87-MG cells were mechanically dispersed, washed, and resuspended in PBS. At t=2-3 min, the osmolality of the bathing medium was increased to 450 mOsM/kg by the addition of NaCl from a 3M stock solution. The time course of volume recovery was continuously followed by Coulter counter analysis in the absence (control) or presence of 80 nM PcTX1 or scrambled PcTX1 peptide.

FIG. 26 shows the effect of PcTX1 on cell growth.

FIGS. 27A-C show the effect of PcTX1 on the growth of U251-MG brain tumors is SCID mice. SCID mice were implanted with U251-MG cells and treated with either saline (27A, upper panels), scrambled peptide (27B, middle panels), or PcTX1 (27C, lower panel). After sacrifice, brain tissue was removed, embedded with paraffin and sectioned (10 μm thick). Sections were stained using hemotoxylin and eosin. Magnifications are 1×, 4× and 20× as indicated.

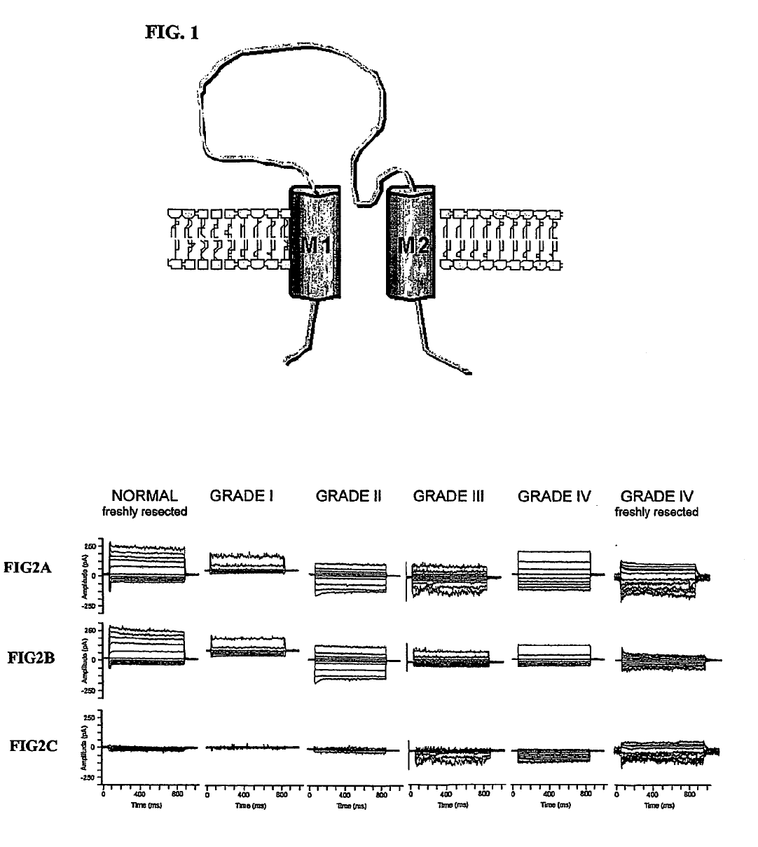

### DETAILED DESCRIPTION

It has been observed that ion channels may be intimately involved in the cellular pathophysiology of cancer. Several different laboratories have demonstrated that the expression of certain oncogenes directly affect sodium (13-15), potassium (16-19), and calcium (13,20,21) channel function. For example, the ras oncogenes, known to be involved in metastasis (22), influence nerve growth factor induced neuronal differentiation and voltage sensitive sodium channel expression and calcium currents (21,23,24). Moreover, cell adhesion (25), motility (26,27), interaction with extracellular matrix (28), and proliferation (13,19,29-31) are all intimately linked to ion channel activity. Therefore, inhibition of ion channel activity serves as a point for pharmacological inhibition of the cellular pathophysiology of cancers.

The present disclosure is directed to the description of a constitutive amiloride-sensitive inward Na⁺ current that is associated with various tumor types and carcinogenesis in a variety of mammalian cell types. The ion channel mediating the inward Na⁺ current is also described. In one embodiment, the ion channel mediating the inward Na⁺ current comprises an ASIC component, such as an ASIC1 component. In an alternate embodiment, the ion channel mediating the inward Na⁺ current may lack a functional ASIC2 component. The constitutive inward Na⁺ current is associated with tumor cell invasion, tumor cell volume recovery after cell shrinkage and tumor cell proliferation. Therefore, inhibition of this constitutive inward Na⁺ current serves as a point for pharmacological intervention in the treatment of carcinogenesis.

Described herein are methods of treating tumors characterized by the expression of a constitutive inward Na⁺ current mediated by a Na⁺ channel containing an ASIC component, such as an ASIC1 component. Methods for the diagnosis/identification of tumors characterized by the expression of a constitutive inward Na⁺ current are described. Methods for visualization of such tumors are also provided. In addition, methods for screening and identification of novel therapeutic agents useful in the treatment of disease states expressing a constitutive inward Na⁺ current are described. The present disclosure describes in detail the application of these teachings to glial-derived tumors, such as gliomas. However, the teachings of the present disclosure are applicable to any tumor characterized by the expression of a constitutive inward Na⁺ current mediated by a Na⁺ channel having an ASIC component Such tumors include, but are not limited to, glioma, breast cancer and melanoma.

Glial-derived tumors comprise a diverse group of neoplasms that differ in their morphology, their CNS location, their degree of invasiveness, their tendency for progression, and their growth characteristics. Neoplastic transformation can occur in all glial cell types, thereby producing a large range of pathological and morphological variants. Most primary brain tumors derived from glial cells that have lost growth control regulation, giving rise to astrocytomas, glioblastomas, or oligodendrocytomas. High-grade gliomas account for 30% of primary brain tumors in adults, and are the second most common cause of cancer death in children under 15 years of age (8,9). High-grade gliomas are divided by grade into two categories: anaplastic astrocytomas (WHO Grade III) and glioblastoma multiforme (GBM; WHO Grade IV) (10). There are also two other histopathologically classified grades of brain tumors, namely, Grades I and II. Increasing grades represent increasing malignancy and decreasing differentiation, which is associated with increased mitotic activity and enhanced cell migration (11,12). Thus, glioma cells exhibit a remarkable degree of heterogeneity that includes not only histological and karyotypic features, but changes in cell motility and selective alterations and cellular oncogenes and tumor suppressor genes.

In spite of this high degree of heterogeneity of gliomas, in all cells isolated from biopsy material obtained from patients who were diagnosed with high-grade gliomas, the presence of a novel, constitutive, amiloride-sensitive, inward Na⁺ conductance was observed. This constitutive, amiloride-sensitive, inward Na⁺ conductance was not present in normal glial cells or in WHO Grade I and II stage tumors. The presence of this amiloride-sensitive, inward Na⁺ conductance persisted in primary cultures of cells derived from high-grade gliomas, as well as continuous cell lines that were originally derived from GBMs. Molecular biological, immunocytochemical, and pharmacological data suggest that the ion channels mediating the inward Na⁺ current may be comprised of subunits of the Deg/ENaC superfamily of ion channels, such as ASIC and ENaC subunits, as wells as other subunits. This suggests that the constitutive amiloride-sensitive, inward whole-cell Na⁺ currents may be a selective property of high-grade glial-derived tumors and other tumor types, such as breast tumors and melanomas.

As described in the present disclosure, all high-grade glioma cells, derived either from freshly resected tumors or from established cell lines, express a constitutively active, amiloride-sensitive inward Na⁺ current. This inward Na⁺ current is important in the proliferation and invasiveness of tumor cells. In contrast, this constitutively active, amiloride-sensitive inward Na⁺ conductance can not be detected in astrocytes obtained from normal brain tissue or from glioma cells derived from low-grade or benign tumors. Constitutive, amiloride-sensitive inward Na⁺ currents have also been detected by Applicants in breast cancer and melanoma cells.

Methods of Treatment

The present disclosure provides for methods of treating tumors characterized by the expression of a constitutive inward Na⁺ current mediated by a Na⁺ channel containing an ASIC component, such as an ASIC1 component The tumor may be derived from glial cells, epithelial cells, melanocytes or other cell types. The tumors derived from glial cells may be gliomas, such as, but not limited to, astrocytomas, glioblastomas and medulloblastomas. The tumors derived from epithelial cells may be breast carcinomas. The tumors derived from melanocytes may be melanomas. Given the teachings of the present disclosure, one of ordinary skill in the art could identify other tumor types expressing such a constitutive inward Na⁺ current.

In one embodiment, the method of treating involves administering to a subject in need of such treatment a therapeutically effective amount of a pharmaceutical composition containing a compound that inhibits the activity of the Na⁺ channel mediating a constitutive inward Na⁺ current. Such a compound may be identified as described below in this specification. Alternatively, such a compound may be PcTX1, or a variant of PcTX1. The inhibition of the Na⁺ channel mediating a constitutive inward Na⁺ current by the compound may be a direct inhibition or indirect inhibition. Direct inhibition may occur by blocking the activity of a component of the Na⁺ channel mediating the constitutive inward Na⁺ current. In one embodiment, the inhibition may occur by blocking the activity of the ASIC component, such as an ASIC1 component Indirect inhibition may occur by blocking an activity required for the activity of the Na⁺ channel mediating the constitutive inward Na⁺. In one embodiment, such activity may be a protein required for the activation of the Na⁺ channel mediating the constitutive inward Na⁺ current or that is involved in the down-regulation of such Na⁺ channel mediating the constitutive inward Na⁺ current, such as a protease or a PKC family members. A “therapeutically effective amount”, in reference to the treatment of a tumor or other disease or condition, refers to an amount of a compound that is capable of having any detectable, positive effect on any symptom, aspect, or characteristics of the tumor or other disease or condition.

In an alternate embodiment, the method of treating involves administering to a subject in need of such treatment a therapeutically effective amount of a pharmaceutical composition containing a compound that binds to the Na⁺ channel mediating the constitutive inward Na⁺ current Such a compound may be identified as described below in this specification. Alternatively, such a compound may be PcTX1, or a variant of PcTX1. Such compound may be linked to a cytotoxic agent The cytotoxic agent may be any agent that is capable of killing or inhibiting the growth of said tumors, such as, but not limited to, a radiolabel, gelonin, ricin, saponin, pseudomonas exotoxin, pokeweed antiviral protein, diphtheria toxin and complement proteins. The radiolabel may be any radialoabel, such as, but not limited to, ¹³¹I and ¹²⁵I. Such binding of the compound to the Na⁺ channel mediating the constitutive inward Na⁺ current may, but is not required to, inhibit the activity of such Na⁺ channel.

Furthermore, the compound may be conjugated to a protein sequence that serves as a protein tag (the tag protein). As above, such compound may be identified as described below in this specification or such compound may be PcTX1, or a variant of PcTX1. In the instance where the compound is PcTX1, or a variant of PcTX1, such PcTX1 or variant of PcTX1 may have a tyrosine residue or other residue at one end thereof to aid in the linking to the tag protein. Such as PcTX1 molecule is shown in SEQ ID NO. 2 and has been shown to have activity identical to the unmodified PcTX1 sequence. In this embodiment, the method of treatment further includes administering to the subject a therapeutically effective amount of a second compound which binds to the tag protein. The second compound may be an antibody, such as a monoclonal antibody. The second compound may be fused to a cytotoxic agent. The cytotoxic agent may be any agent that is capable of killing or inhibiting the growth of said tumors, such as, but not limited to, a radiolabel, gelonin, ricin, saponin, pseudomonas exotoxin, pokeweed antiviral protein, diphtheria toxin and complement proteins. The radiolabel may be any radiolabel, such as, but not limited to, ¹³¹I and ¹²⁵I. In a specific example, the compound may be PcTX1 and the tag protein may be glutathione-S-transferase; the second compound may be a monoclonal antibody recognizing said glutathione-S-transferase that is fused to a cytotoxic agent.

Psalmotoxin 1 (PcTX1) is a peptide isolated from the venom of the South American tarantula Psalmopoeus cambridgei. PcTX1 is a 40 amino acid peptide possessing 6 cysteine residues linked by three disulfide bridges. The amino acid sequence of PcTX1 is shown in SEQ ID NO: 1. PcTX1 has a limited homology with other spider toxins known in the art However, PcTX1 does share a conserved cysteine distribution found in both spider and cone snail peptide toxins (64). As used in the present disclosure, PcTX1 is defined as the peptide the amino acid composition of which is shown in SEQ ID NO: 1 or SEQ ID NO. 2. The present disclosure is also directed to variants of PcTX1 that retain the activity of the peptide disclosed in SEQ ID NO: 1 or SEQ ID NO. 2. Generally, differences are limited so that the sequences of the reference polypeptide and the variant are closely similar overall and, in many regions, identical. A variant and reference polypeptide may differ in amino acid sequence by one or more substitutions, additions, deletions in any combination. A variant may be a naturally occurring or it may be a variant that is not known to occur naturally. Non-naturally occurring variants of may be made by mutagenesis techniques or by direct synthesis. A variant may also include conservative amino acid substitutions. PcTX1 also includes fragments of the polypeptide shown in SEQ ID NO: 1 or SEQ ID NO. 2, where said fragments are at least five amino acids in length. In one embodiment, the fragment of PcTX1 contains all six cysteine residues. PcTX1 or a variant of PcTX1 may be purified from natural sources, may be produced synthetically, or may be produced as a recombinant protein from genetically engineered cells. In one embodiment, PcTX1 or a variant of PcTX1 is used in a purified form. In an alternate embodiment, PcTX1 r a variant of PcTX1 is used in a partially purified form.

Pharmaceutical compositions of the present disclosure containing the compounds discussed above, such as, but not limited to, PcTX1 may be formulated in combination with a suitable pharmaceutical carrier for administration to a subject in need of treatment. Such pharmaceutical compositions comprise a therapeutically effective amount of the polypeptide or compound, and a pharmaceutically acceptable carrier or excipient Such carriers include but are not limited to, saline, buffered saline, dextrose, water, glycerol, ethanol, and combinations thereof. Formulation should suit the mode of administration, and is well within the skill of the art The invention further relates to pharmaceutical packs and kits comprising one or more containers filled with one or more of the ingredients of the aforementioned compositions of the invention. Compounds of the present invention may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, intracranial or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible. Administration of these compounds may also be topical and/or localized, in the form of salves, pastes, gels and the like. The dosage range required depends on the choice of peptide, the route of administration, the nature of the formulation, the nature of the subject's condition, and the judgment of the attending practitioner. Suitable dosages, however, are in the range of 0.1-100 pg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

In still another approach, expression of the gene encoding a component of the Na⁺ channel mediating the constitutive, amiloride-sensitive inward Na⁺ current can be inhibited using expression blocking techniques. Known techniques involve the use of antisense sequences, either internally generated or separately administered. See, for example, O'Connor, J Neurochem (1991) 56:560 in Oligodeoxynucleotides as Antisense Inhibitors of Gene Expression, CRC Press, Boca Raton, Fla. (1988). Alternatively, oligonucleotides which form triple helices with the gene can be supplied. See, for example, Lee et al., Nucleic Acids Res (1979) 6:3073; Cooney et al., Science (1988) 241:456; Dervan et al., Science (1991) 251:1360. These oligomers can be administered per se or the relevant oligomers can be expressed in vivo. Non-coding RNAs (ncRNA) (also referred to as functional RNA, or fRNA), such as miRNA (microRNA), rRNA (ribosomal RNA), siRNA (small interfering RNA), snRNA (small nuclear RNA), snmRNA (small non-mRNA), snoRNA (small nucleolar RNA) and stRNA (small temporal RNA), may also be used to block the expression of a gene encoding a component of the Na⁺ channel.

Polypeptides used in treatment can also be generated endogenously in the subject, in treatment modalities often referred to as “gene therapy”. Thus, for example, cells from a subject may be engineered with a polynucleotide, such as a DNA or RNA, to encode a polypeptide ex vivo, and for example, by the use of a retroviral plasmid vector. The cells are then introduced into the subject In one embodiment, the cells express PcTX1.

Method of Diagnosis

The teachings of the present disclosure may be used to identify and/or diagnose individuals with a tumor characterized by a Na⁺ channel mediating a constitutive inward Na⁺ current. The tumor may be derived from glial cells, epithelial cells, melanocytes or other cell types. The tumors derived from glial cells may be gliomas, such as, but not limited to, astrocytomas, glioblastomas and medulloblastomas. The tumors derived from epithelial cells may be breast carcinomas. The tumors derived from melanocytes may be melanomas. In one embodiment the method of identification and/or diagnosis relies on the identification of a constitutive, amiloride-sensitive, inward Na⁺ conductance in the tissue to be tested. In an alternate embodiment the method of identification and/or diagnosis relies on the absence or presence of a component of the Na⁺ channel mediating a constitutive inward Na⁺ current, in the tissue to be tested. In one embodiment, the method may rely on the detection of the ASIC1 component Detection may occur at the protein or nucleic acid level. In an alternate embodiment, the method may rely on the lack of detection of a functional ASIC2 component. Detection may occur at the protein or nucleic acid level. Such methods are well known in the art

In one embodiment, the method of diagnosis/identification involves administering to a subject in need of such diagnosis/identification diagnostically effective amount of a reagent that recognizes a component of the channel responsible for the constitutive, amiloride-sensitive, inward Na⁺ conductance and measuring the level of binding of the reagent in said subject Such a reagent may be identified as described below in this specification. Alternatively, such a reagent may be PcTX1, or a variant of PcTX1. A “diagnostically effective amount”, in reference to the diagnosis/identification of a tumor or other disease or condition, refers to an amount of a reagent that on interacting with said Na⁺ channel is capable of being detected by current detection methodologies. A positive diagnosis/identification indicates the subject may have a tumor characterized by said Na+ channel mediating a constitutive inward Na+ current. The subject may undergo additional testing or may begin therapeutic treatment

In one embodiment, the reagent may be a polypeptide capable of binding a component of the ion channel responsible for the constitutive, amiloride-sensitive, inward Na⁺ conductance. In one embodiment, the polypeptide may be the PcTX1 toxin or a variant of the PcTX1 toxin The polypeptide may be conjugated to a diagnostic label capable of detection by imaging methods known in the art. The diagnostic agent may be a fluorescent agent, a radiolabel, a luminescent agent or other agent capable of being detected by current detection methodologies, such as MRI or CT methodology. The radiolabel may be any radiolabel, such as, but not limited to, ¹³¹I and ¹²⁵I.

Furthermore, the polypeptide may be conjugated to a protein sequence that serves as a protein tag (the tag protein). The polypeptide may be PcTX1, or a variant of PcTX1. In the instance where the compound is PcTX1, or a variant of PcTX1, such PcTX1 or variant of PcTX1 may have a tyrosine residue or other residue at one end thereof to aid in the linking to the tag protein. Such as PcTX1 molecule is shown in SEQ ID NO. 2 and has been shown to have activity identical to the unmodified PcTX1 sequence. In this embodiment, the method of diagnosis/identification further includes administering to the subject a diagnostically effective amount of a second compound which binds to the tag protein. The second compound may be an antibody, such as a monoclonal antibody. The second compound may be fused to a diagnostic agent. The diagnostic agent may be a fluorescent agent, a radiolabel, a luminescent agent or other agent capable of being detected by current detection methodologies, such as MRI or CT methodology. The radiolabel may be any radiolabel, such as, but not limited to, 131I and 125I. In a specific example, the polypeptide may be PcTX1 and the tag protein may be glutathione-S-transferase; the second compound may be a monoclonal antibody recognizing said glutathione-S-transferase that is fused to a diagnostic agent

In another embodiment, the reagent used is an antibody. The antibody may be polyclonal or monoclonal antibodies, or any fragment thereof capable of binding (such as, but not limited to Fab₂ fragments) to the Na⁺ channel mediating the constitutive inward Na+ current or a component thereof. The component may be an ASIC component, such as ASIC1. The antibody may be fused to a diagnostic agent. The diagnostic agent may be a fluorescent agent, a radiolabel, a luminescent agent or other agent capable of being detected by current detection methodologies, such as MRI or CT methodology. The radiolabel may be any radiolabel, such as, but not limited to, ¹³¹I and ¹²⁵I.

In an alternate embodiment, the reagent may be a nucleic acid molecule, such as a primer for PCR or RT-PCR reaction. The reagent may further comprise a detection molecule. Such detection molecules are well known in the art and may be a radiolabel, a fluorescent label or an enzymatic label.

In one application, the reagent is administered to a subject prior to or at the time of a surgical procedure. The reagent may be visualized during the surgical procedure to aid in the identification of the tumor tissue and serve as a guide to the healthcare provider in identifying the tumor tissue and removing the tumor tissue. In this case, the diagnostic/visualization agent is one that may be visualized during the surgical procedure. In one specific embodiment, the reagent is PcTX1 or a variant of PcTX1 fused to a diagnostic agent as described above.

Method for Identifying Inhibitors

The teachings of the present disclosure may be used to identify compounds which bind to or inhibit the constitutive, amiloride-sensitive, inward Na⁺ conductance. The inhibition may be direct or indirect. For direct inhibition, the compounds may inhibit the constitutive, amiloride-sensitive, inward Na⁺ conductance by directly inhibiting a component of the channel responsible for mediating the constitutive, amiloride-sensitive, inward Na⁺ conductance. In one embodiment, direct inhibition may occur as a result of compound inhibiting the function of the ASIC1 component. Indirect inhibition may occur by inhibiting a cellular pathway involved in the positive regulation of the constitutive, amiloride-sensitive, inward Na⁺ conductance or activating a cellular pathway involved in the negative regulation of the constitutive, amiloride-sensitive, inward Na⁺ conductance. Suitable pathways include, but are not limited to, those pathways described in the instant disclosure.

In one embodiment, such identification involves a screening assay utilizing a system which incorporates a Na⁺ channel mediating the constitutive, amiloride-sensitive inward Na⁺ current in a functional state. A functional state is defined as any Na⁺ channel comprising a combination of components resulting in a constitutive, amiloride-sensitive inward Na⁺ current. The components may include ASIC components, such as ASIC 1 and ASIC 2, as well as other ENaC/DEG family members and proteins involved in the regulation of any of the foregoing, such as PKC isoforms syntaxin family members, such as syntaxin 1A and proteases, such as MT-SP1 or other members of the TTSP family. The screening assay may utilize lipid bilayers, oocytes, drosophila, yeast, bacterial or mammalian cells expressing the Na⁺ channel mediating the constitutive, amiloride-sensitive inward Na⁺ current in a functional state. Examples of such systems are described herein. Furthermore, membrane preparations or vesicles can be formed from any of the above and used to conduct the identification procedures.

The present disclosure shows that the composition of the Na⁺ channels responsible for mediating the constitutive, amiloride-sensitive, inward Na⁺ conductance is unique in high-grade gliomas. For example, as described in the present disclosure, the channels in high-grade gliomas lack a functional ASIC2 component at the plasma membrane. In one embodiment the functional state may include ASIC1 protein co-expressed with other proteins, such as, but not limited to γENaC, PKC family members or proteases, such as members of the TTSP family. Other proteins that may be co-expressed with ASIC1 are known in the art and described in the present disclosure in the section titled “Examples.” In addition, the functional state may include certain mutations to ASIC1, such as, but not limited to, the G433F mutation. In an alternate embodiment, the functional state may lack ASIC2 protein or nucleic acid.

An appropriate assay utilizing a system which expresses an ion channel mediating the constitutive, amiloride-sensitive inward Na⁺ current in a functional state as described above is contacted with a test compound to observe binding to, or modulation of a functional response of said Na⁺ channel. Modulation of a functional response may include activation or inhibition of the constitutive, amiloride-sensitive, inward Na⁺ conductance and the activation or inhibition of signaling events triggered by the activation or inhibition of the constitutive, amiloride-sensitive, inward Na⁺ conductance or which modulate the activity of said Na⁺ channel. Test compounds may be polypeptides, organic molecules, inorganic molecules, small molecules, substrates and ligands. The functional response may be monitored by any of the methods described in the present disclosure or other methods known in the art. In a binding assay, the assay may simply test binding of a test compound to said Na⁺ channel, wherein adherence to said Na⁺ channel is detected by means of a label directly or indirectly associated with the test compound. Alternatively, the assay may involve competition with a labeled competitor. Standard methods for conducting such screening assays are well understood in the art.

### EXAMPLES

### Example 1A

### Grade III and IV Human Gliomas Express a Constitutive Inward Na⁺ Conductance that is Sensitive to Amiloride

A constitutive amiloride-sensitive inward Na⁺ conductance has been reported in human high-grade glioma cells. These inward Na⁺ currents were seen in primary cultures of freshly resected high-grade gliomas as well as in established cell lines derived from high-grade gliomas. These inward Na⁺ currents were not present in normal astrocytes or in low-grade astrocytomas (e.g., pilocytic astrocytomas). However, the composition of the channels responsible for the inward Na⁺ conductance has not been reported. FIGS. 2A-C show representative whole cell patch-clamp measurements on tissue derived from a freshly resected human glioblastoma multiforme (GBM; WHO grade IV), normal astrocytes obtained from patients undergoing surgery for intractable epilepsy, and primary cultures of different grade glial tumors. In the basal state, the current records for both freshly resected and primary cultured Grade III and IV tumor cells were characterized by large inward currents (FIG. 2A), and these currents were completely inhibited following superfusion with 100 μM amiloride (FIG. 2B). Panel C of FIG. 2 shows the difference current (i.e., the amiloride-sensitive component). Grade III and IV tumor samples showed a significant amiloride-sensitive component. However, there was no significant inward Na⁺ current in normal astrocytes and Grades I and II astrocytoma cells (FIG. 2C). These results suggest the contribution of an amiloride-sensitive component to the inward Na⁺ currents only in the high-grade gliomas.

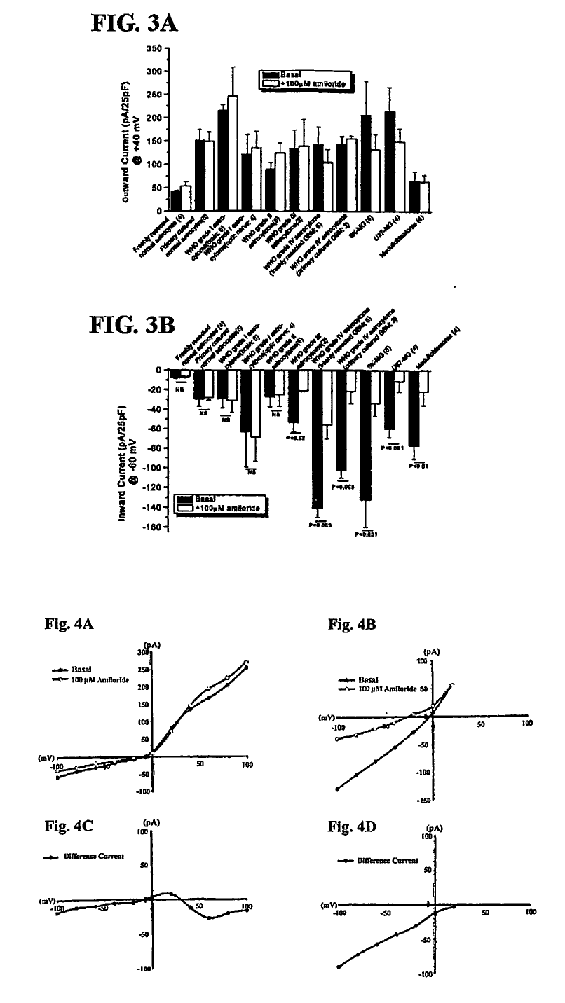

The absolute magnitudes of the outward currents at +40 mV (FIG. 3A) and inward currents at −60 mV (FIG. 3B) in the absence and presence of amiloride for normal astrocytes, different grade gliomas, medulloblastoma, and two GBM continuous cell lines are summarized. While there is no discernible pattern to the magnitudes of either the outward or inward currents, amiloride only blocked inward Na⁺ currents in the high-grade gliomas (Grades III and IV, and medulloblastoma) consistent with the results above. Amiloride likewise blocked inward Na⁺ currents in SK-MG and U87-MG cells, both originally derived from GBM.

Summary current-voltage (I-V) curves are presented for normal astrocytes and GBM cells in FIG. 4A and FIG. 4B, respectively, while difference I-V curves for normal astrocytes and GBM cells are presented in FIG. 4C and FIG. 4D, respectively. The GBM cells are depolarized by an average of 31 mV compared to the normal astrocytes under these recording conditions. The depolarized zero current membrane potential is due to the presence of an enhanced Na⁺ conductance as is shown in the difference I-V curves. As before, 100 μM amiloride did not affect currents in normal astrocytes (FIG. 4C), but significantly inhibited inward Na⁺ currents in GBM (FIG. 4D). The reversal potential of the GBM shifted in the hyperpolarizing direction in the presence of amiloride, and the amiloride-sensitive current reversed at ˜+30 mV, indicating that this current was carried primarily by Na⁺.

### Example 1B

### Breast Carcinoma and Melanoma Cells Express a Constitutive Inward Na⁺ Conductance that is Sensitive to Amiloride

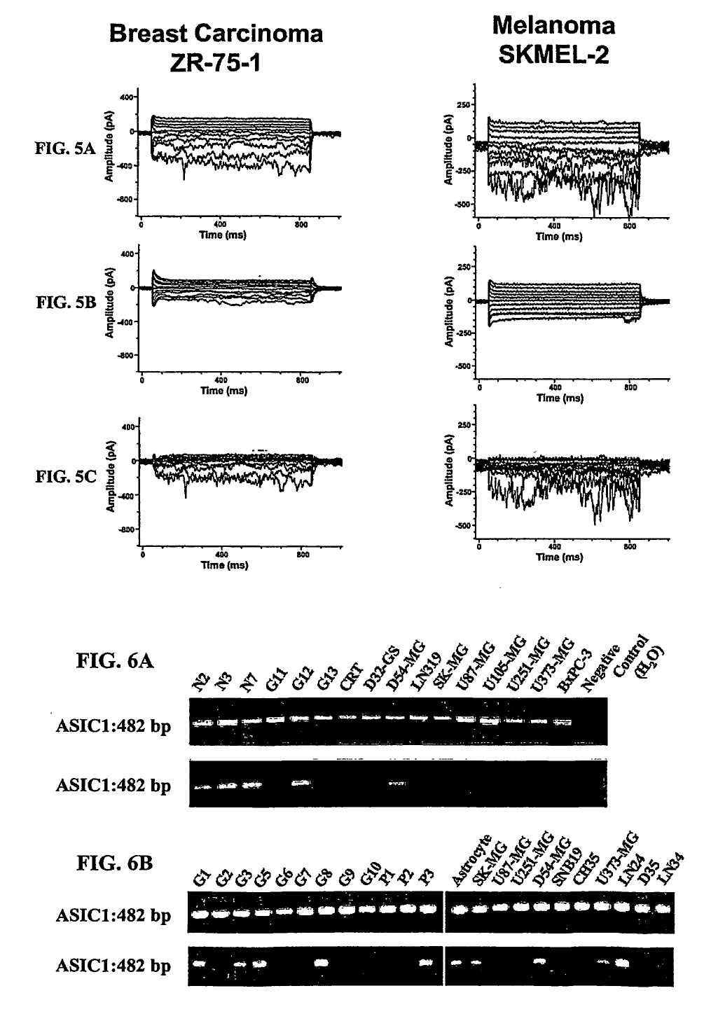

A constitutive amiloride-sensitive inward Na⁺ conductance was also observed in the breast carcinoma cell line ZR-75-1 and the melanoma cell line SKMEL-2. FIGS. 5A-C show representative whole cell patch-clamp measurements on ZR-75-1 cells and SKMEL-2 cells. In the basal state, the current records for both tumor cell lines were characterized by large inward currents (FIG. 5A), and these currents were completely inhibited following superfusion with 100 μM amiloride (FIG. 5B). FIG. 5C shows the difference current (i.e., the amiloride-sensitive component). Both ZR-75-1 and SKMEL-2 cells showed a significant amiloride-sensitive component These results suggest the contribution of an amiloride-sensitive component to Na⁺ currents only in multiple types of cancers.

### Example 2

### ASIC Components are Involved in the Inward Sodium Conductance Observed in Glial Cells

RT-PCR was performed on total RNA extracted from human tissue samples obtained during craniotomy for epilepsy (normal tissue, labeled N in top panel) or for primary GBM resections (lanes labeled G), primary normal astrocytes, and continuous cell lines derived from an anaplastic astrocytoma (CRT), a gliosarcoma (D32-GS), and fourteen different GBMs (FIGS. 6A and B). Two independent sets of experiments were done.

Specific primers were designed to amplify a 447 bp product of ASIC2 and a 482 bp product for ASIC1. Primers for ASIC1 spanned bp 1091-1537 and primers for ASIC2 spanned bp 1109-1587+3′ UTR. All reactions were negative for genomic DNA (i.e., PCR without added RT). A (ΦX174 HaeII molecular weight ladder was used for size determination and PCR products were resolved using a 2% NuSieve agarose gel. The ASIC1 product was detected in all of the samples, both normal and tumor, including a pancreatic carcinoma cell line (FIG. 6A, BxPc3). ASIC1 product was not contained in the negative control lanes. In contrast, the ASIC2 message was found in the four normal samples, (N2, N3, N7), astrocytes (FIG. 6B), and in 6/15 freshly resected and primary GBMs (G1, G3, G5, G8, G12, P3, FIGS. 6A and B) and in 4/12 GBM cell lines (D54-MG, SK-MG, U373-MG, and LN24, FIGS. 6A and B). Direct sequencing of the PCR products confirmed their identity. These results show that ASIC1 is present in both normal astrocytes and GBM tissues, and ASIC2 can be detected in normal astrocytes, but not in the majority of high-grade gliomas (60-70%).

### Example 3

### Relationship Between ASIC Expression and Inward Na⁺ Current

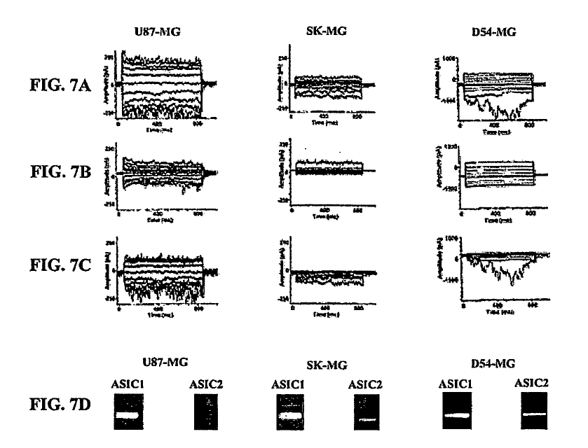

The amiloride-sensitive inward Na⁺ currents are measured regardless of whether ASIC2 is absent or present (FIGS. 7A-D). Whole-cell patch clamp recordings were obtained from U87-MG, SK-MG, and D54-MG glioma cells in the basal state. Amiloride (100 μM) inhibited inward currents in all three cell types (as can be seen in the difference current tracings)(FIG. 7A-C). This inhibition of the inward current occurred regardless of the absence or presence of ASIC2 mRNA (FIG. 7D) (as detected by RT-PCR as described above). As can be seen, amiloride inhibited inward conductance in U87-MG cells, where ASIC2 mRNA is absent, as well as in SK-MG and D54-MG cells where ASIC2 mRNA is present. While not being limited to other theories, the data suggests that only ASIC1 is present in the plasma membrane and in those cells containing ASIC2 mRNA and protein, the ASIC2 protein may remain intracellular.

### Example 4

### ASIC1 and ASIC2 Interaction in Oocytes Alter the Conductance Characteristics of the Individual Channels Mediating the Inward Na⁺ Current

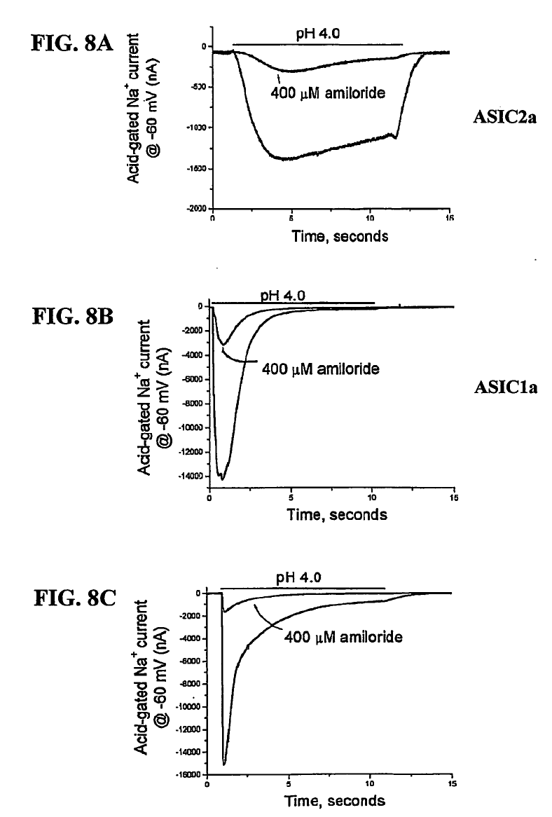

Xenopus oocytes were used to express ASIC1 and ASIC2 mRNA individually and in combination. Oocyte preparation, cRNA injection, and two-electrode voltage clamp recordings were performed as described (33,67-69). FIGS. 8A-C show amiloride-sensitive, inward Na⁺ currents (at −60 mV) in cells expressing either ASIC1 or ASIC2 alone, or the combination of ASIC1 and ASIC 2 in individual voltage-clamped oocytes activated by decreasing extracellular pH from 7.5 to 4.0 (solid bar) using a gravity-fed rapid superfusion system. FIG. 8A shows ASIC2 expression, FIG. 8B shows ASIC1 expression and FIG. 8C shows the combination of ASIC1 and ASIC2 expression. There is no measurable current when the extracellular pH (pHₒ) is 7.2. However, upon reduction of pHₒ to 4.0 (indicated by the bar in each Panel), there is a transient current response comprised of two currents: the peak inward current (1p) and the steady-state current (Is), which was measured approximately 8 s after Ip. As seen in FIGS. 8A-C, the time course of acid-activated ASIC2 current is slower and more pronounced than that of ASIC1 or for the combination of ASIC1/2, especially following Ip. All acid-activated currents were inhibited by 400 μM amiloride, a maximally inhibitory concentration of drug. Ip was greatest for ASIC1, and least for ASIC2. This data suggests that ASIC1 and ASIC2 induce cation-selective currents, with the P_{Na}/P_{K} ranging from 2 to 4 (I-V curves not shown), and that ASIC1 and ASIC2 interact in such a way that the conductance characteristics of the individual channels are altered.

Wild-type ASIC1 incorporated into planar lipid bilayers at neutral pH or at acidic extracellular pH was also examined. When bathed in 100 mM NaCl, these channels displayed a conductance of 20 pS, and at neutral pH were only open an average 8% of the time. However, lowering the trans solution pH to 6.2 caused the channels to remain open greater than 90% of the time (Po=0.9±0.08, N=10). Amiloride produced a flickery-block of the channel, consistent with its effects on other members of the Deg/ENaC family (1-3). At pH 7.4, the apparent equilibrium inhibitory dissociation constant (Kᵢ) of amiloride was 0.82±0.09 μM (N=7). At pH 6.2, the curve was slightly right-shifted, the Kᵢ being 2±0.23 μM (N=6).

The characteristics of mutant ASIC1 were also examined. ASIC1 nucleic acid was modified to substitute phenylalanine for glycine at position of 433 of ASIC1. This mutation has previously been shown to activate ASIC1 channels (64-66) and have been shown to produce neurodegeneration in C. elegans (4-5 and 70-71). The mutated ASIC1 channel showed constitutive activation of the channel, increasing Po from 0.08±0.03 in the wild-type channel to 0.89±0.09 (N=5). The channel's sensitivity to amiloride was slightly right shifted as compared to the wild-type (2.5 μM vs. 0.8 μM at pH 7.4).

### Example 5

### ASIC1 and ASIC2 Are Capable of Forming Heteromeric Complexes

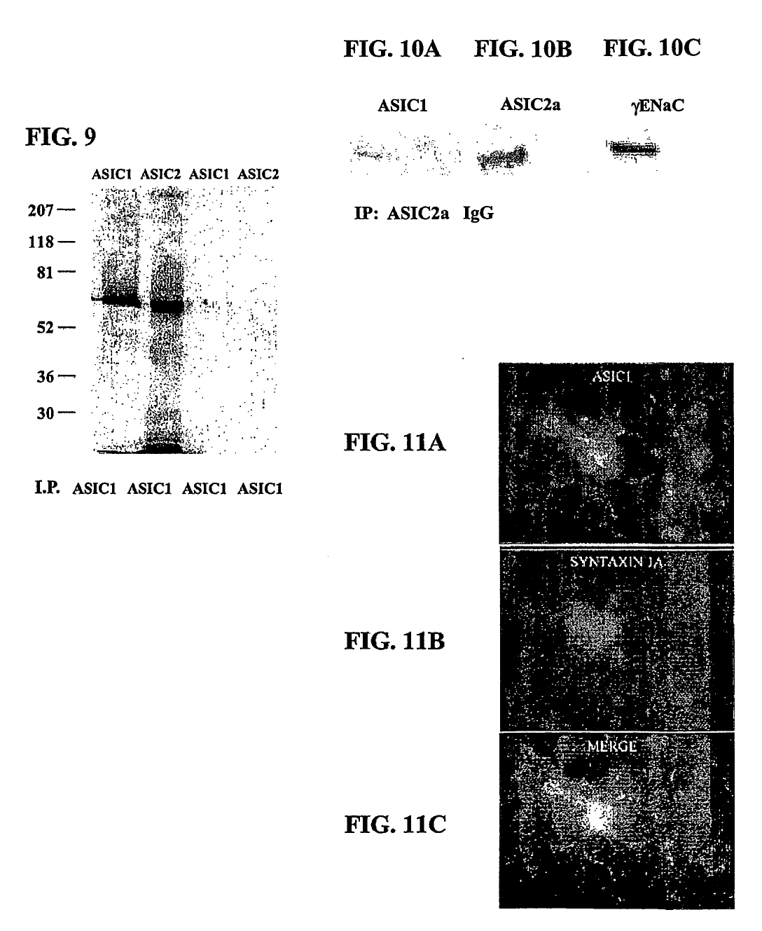

The experiments in FIG. 9 show that ASIC1 and ASIC2 are capable of interaction and that this interaction alters the conductance characteristics of the channels mediating the constitutive amiloride-sensitive inward Na⁺ current To examine this interaction, proteoliposomes containing in vitro translated [³⁵S] methionine labeled ASIC1 or ASIC2 plus the unlabeled conjugate partner were produced. FIG. 9 shows that immunoprecipitation of the unlabeled conjugate partner also immunoprecipitated the labeled conjugate partner as determined by autoradiography. In the left-most lane, protein from proteoliposomes containing ³⁵[S]-met ASIC1 plus unlabeled ASIC2 was immunoprecipitated using anti-ASIC2 antibodies. ASIC 1 protein is detected. The next lane shows that ASIC2 is present in the immunoprecipitate of a mixture of ³⁵[S]-met ASIC2 plus unlabeled ASIC1 immunoprecipitated using anti-ASIC1 antibodies. The last two lanes demonstrate that antibodies against ASIC2 or ASIC1 cannot immunoprecipitate radioactively-labeled ASIC1or ASCI2, respectively.

Co-immunoprecipitation experiments were also performed in the tumor cell line, SK-MG (FIGS. 10A-C). SK-MG cells express an amiloride-sensitive, inward Na⁺ current and as determined by RT-PCR, contain message for ASIC1, ASIC2, ASIC3, and γ-hENaC Using anti-ASIC2 antibodies as the precipitating agent, ASIC1 (FIG. 10A), ASIC3 (FIG. 10B), and γ-hENaC (FIG. 10C) can all be detected in the precipitate. Control immunoprecipitates using IgG were negative for all of the above (FIGS. 10A-C). These results suggest that multiple ASIC and ENaC components co-exist in a multimeric complex.

This finding was confirmed by immunolocalization studies. SK-MG cells were grown on chamber slides. Cells were fixed with 4% formaldehyde in PBS, permeabilized with 0.1% Triton X-100 in PBS, and blocked with 5% normal goat serum in PBS for 120 min. The cells were incubated with the primary antibody solution for 72 h at 4° C. with 5% normal goat serum and 0.1% Triton X-100. Primary antibodies were used in the following dilutions: 1:200 for anti-Syntaxin 1A antibodies, 1:20 for anti-ASIC2a antibodies or anti-ASIC1, and 1:100 for anti-γ-ENaC antibodies. Cells were labeled with one (for single staining) or two (for double staining) secondary antibodies. The samples were rinsed in PBS and exposed to one or two of the following secondary antibodies: goat anti-rabbit-Alexa 594 (1:200) and/or goat anti-mouse-Alexa 488 (1:200) for 2 h at room temperature. Cells were washed five times with PBS and mounted with 50% glycerol. Samples were examined using an Olympus IX 70 fluorescence microscope.

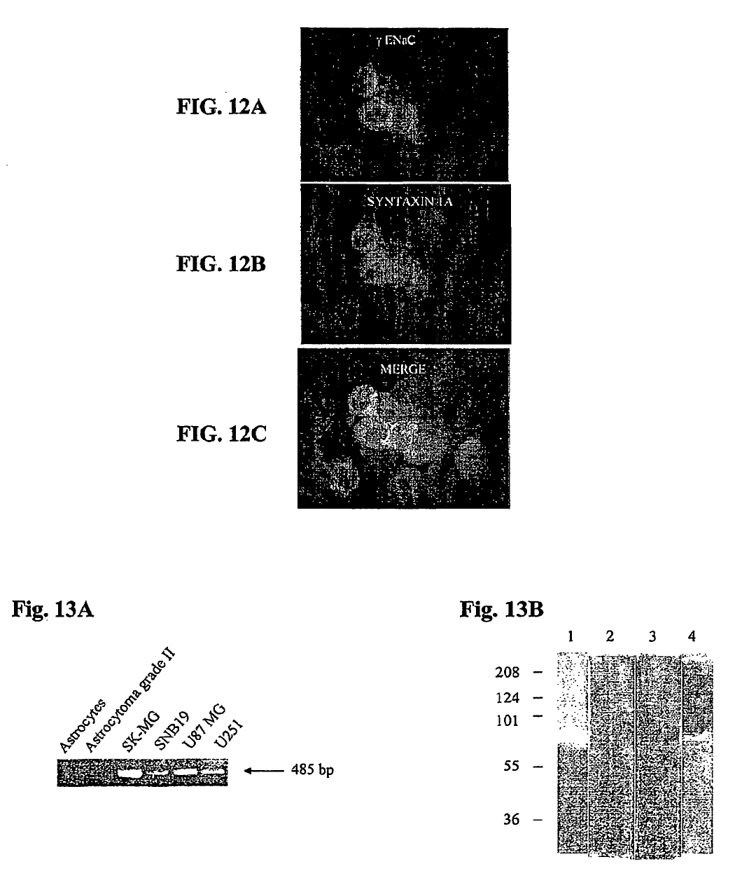

FIGS. 11A-C show co-localization of syntaxin 1A and ASIC1 in SK-MG cells. All of the panels represent epifluorescent images. FIG. 11A shows ASIC1 staining using commercially available polyclonal anti-ASIC1 antibodies (Chemicon). FIG. 11B shows Syntaxin 1A staining using highly specific monoclonal antibodies (no cross reactivity between syntaxin 1A and syntaxin 1B). FIG. 11C shows double staining with anti-syntaxin 1A and anti-ASIC1 antibodies. Overlap is observed, as indicated by yellow. FIGS. 12A-C shows co-localization of syntaxin 1A and γ-hENaC in SK-MG cells. As above, all of the panels represent epifluorescent images. FIG. 12A shows γ-hENaC staining using a commercially available antibody (source). FIG. 12B shows syntaxin 1A staining as described in FIG. 11. FIG. 12C shows double staining with anti-syntaxin 1A and anti-γ-hENaC antibodies. Overlap is observed, as indicated by yellow. These preliminary results are consistent with the findings of others (5,43), and support the hypothesis that ASIC1, syntaxin 1A, and γ-hENaC may physically interact and thus form part of a large macromolecular complex.

Other cellular pathways may also influence the constitutive amiloride-sensitive inward Na⁺ current These pathways may be needed for the proper regulation, either positive or negative, of Na⁺ current activity. Proteases have been known to be involved in cellular carcinogenesis in a wide variety of cell types. The expression of virtually all type II transmembrane serine proteases (TTSPs) characterized to date is widely deregulated (increased) during the development and progression of the tumor processes. This class of cell surface proteolytic enzymes contains a C-terminal extracellular serine protease domain and is ideally positioned to interact with other proteins on the cell surface as well as soluble proteins, matrix components, and proteins on adjacent cells (127). There are no reports about expression of any member of TTSPs in malignant gliomas. The current disclosure shows that the expression of one TTSP family member, matriptase (MT-SP1), correlates with the presence of the constitutive amiloride-sensitive inward Na⁺ current. MT-SP1 in expressed in several glioma cell lines as confirmed by RT-PCR (FIG. 13A). MT-SP1 was detected using RT-PCR with the primers 5′-cacaaggagtcggctgtgac-3′ (SEQ ID NO: 4) and 5′-ggagggtaggtgccacacaa-3′ (SEQ ID NO: 5). RT-PCR products were resolved on a 2% NuSieve agarose gel. Only product of expected size (485 bp) was obtained. MT-SP1 is secreted by SK-MG glioma cell line as shown by gelatin zymography (FIG. 13B). One ml of a 50-fold concentrated conditioned medium from SK-MG cells was subjected to gelatin zymography. After SDS-PAGE, the gel was incubated with different protease inhibitors From left to right, lane 1 served as a control; lane 2, indicates treatment with 10 mM EDTA; lane 3 indicates treatment with 10 mM Aprotinin; and lane 4 indicates treatment with 10 mM of Galardin (Sigma-Aldrich), matrix metalloproteinase inhibitor. After overnight incubation at pH 7.5, proteolytic activities were visualized by Coomassie Brilliant Blue staining. MT-SP1 was originally identified in breast cancer cells and is highly expressed in breast, prostate and colorectal cancers (128-131). Although human breast cancer cells produce MT-SP1 primarily as the free enzyme, in human milk and normal tissues, the enzyme is found in complex with an inhibitor called hepatocyte growth factor activator inhibitor 1 (HAI-1) (132). Inhibition of MT-SP1 abolishes both primary tumor growth and metastasis in a murine model of prostate cancer (130,133), whereas stabilization of active MT-SP1 through glycosylation by N-acetylglucosaminyl-transferase V is associated with the prometastatic effects of this enzyme (134). Therefore, TTSP family members may be involved in the regulation of the constitutive amiloride-sensitive inward Na⁺ current observed.

The involvement of PKC and its isoforms in the regulation of constitutive amiloride-sensitive inward Na⁺ current has been described. RT-PCR evaluation of PKC isoform expression at the level of mRNA revealed the presence of α and ε/ε′ in all glioma cell lines analyzed; most, but not all cell lines also expressed δ and ζ. No messages were found for the βI and βII isotypes of PKC in the high-grade glioma cells. Normal astrocytes expressed PKCβ but not PKCγ. The essential features of these results were confirmed at the protein level by Western analysis. This disproportionate pattern of PKC isoform expression in glioma cell lines was further echoed in the functional effects of these PKC isoforms on ASIC1 activity in bilayers. PKC holoenzyme or the combination of PKCβI and PKCβII isoforms inhibited ASIC1. Neither PKCε, PKCζ, nor their combination had any effect on ASIC1 activity in bilayers. The inhibitory effect of the PKCβI and PKCβII mixture on ASIC1 activity was abolished by a five-fold excess of a PKCε and PKCζ combination. PKC holoenzyme, PKCβI, PKCβII, PKCδ, PKCε, and PKCζ phosphorylated ASIC1 in vitro. In patch clamp experiments, the combination of PKCβI and PKCβII inhibited the basally activated inward Na⁺ conductance. The variable expression of the PKC isotypes and their functional antagonism in regulating ASIC1 activity support the idea that the participation of multiple PKC isotypes contributes to the overall activity of ASIC1.

Differential gene expression profiling was conducted on three human temporal lobe brain tissue samples (normal) and four primary glioblastoma multiforme (GBM) tumors using Affymetrix® oligonucleotide microarrays. Confirmation of altered gene expression of selected genes was done using RT-PCR, whole-cell patch clamp, and immunohistochemistry. These results show that 1) the expression of α- and β-hENaC is not detectable in either normal or tumor samples; 2) γ-hENaC appears to be present in most of the samples (both normal and tumor); and 3) both syntaxin 1A and SNAP23/25 are present in normal tissue and in GBMs. The presence of syntaxin 1A was confirmed by RT-PCR.

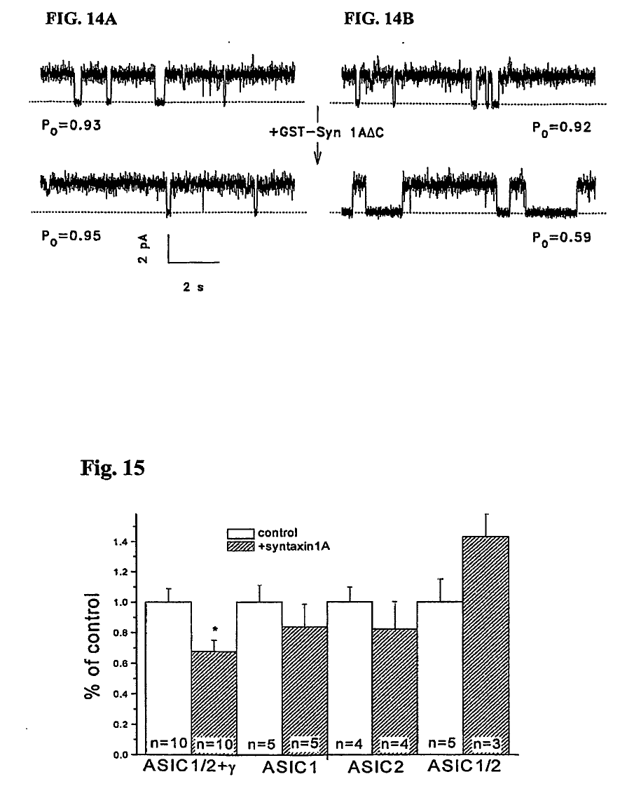

The effect of syntaxin 1A on constitutive amiloride-sensitive inward Na⁺ current activity in planar lipid bilayers was examined (FIGS. 14A and B). For these experiments, membrane vesicles were prepared from oocytes that were previously injected with cRNA encoding both ASIC1 and ASIC2±γ-hENaC. After channel incorporation the extracellular [Ca²⁺] was reduced to <1 nM with EGTA, a condition discovered by one of the Applicants to increase ASIC open probability (Po) and hence produce a continuously active channel in the absence of a gain-of-function mutation or an acid pulse. Syntaxin 1A was then added as a GST fusion protein to the cis (or cytoplasmic) bathing solution. As shown in FIG. 14A, syntaxin 1A had no effect on ASIC1/2 activity. However, when the γ-hENaC subunit was co-expressed with ASIC1/2, 25 μM syntaxin 1A significantly inhibited channel Po by nearly 40% (FIG. 14B). The same results were found in oocyte expression studies (FIG. 15). This effect was specific for syntaxin 1A as syntaxin 3 was without effect

Although normal astrocytes contain the same mRNA as many of the gliomas as determined by RT-PCR (i.e., ASIC1, ASIC2, ENaCs), no constitutive amiloride-sensitive inward Na⁺ current can be measured. Moreover, a sudden drop in external pH from 7.4 to 6.4 does not result in an activation of inward current. While not being limited to alternate explanations, this suggests that, in normal cells, amiloride-sensitive Na⁺ current (and proton-gated) currents may be inhibited by two mechanisms, namely, inhibition by PKC and by syntaxin 1A. In transformed cells, this inhibition fails to occur, resulting in a constitutive inward current This suggest that functional, rather than molecular differences (e.g., mutations) in the channel components are responsible for the constitutively active inward Na⁺ current observed. Syntaxin 1A is expressed in normal cells and gliomas and syntaxin 1A co-localizes both with ASIC 2 and γENaC in SK-MG cells. Furthermore, syntaxin 1A markedly reduces the open probability of heteromeric ASIC1/ASIC2/γENaC channels, but is without effect on the Pₒ of an ASIC1/ASIC2 channel heteromer. These findings are consistent with a model in which a heteromeric channel responsible for the constitutive amiloride-sensitive inward Na⁺ current composed of ASIC1/ASIC2/γENaC is tonically inhibited by interaction cellular factors, such as, but not limited to, syntaxin 1A and PKC, in normal cells (i.e., normal astrocytes). In transformed cells (i.e., high-grade glioma), the heteromeric channel composition is altered such that inward Na⁺ conductance is not inhibited. While not being bound to any one theory, the heteromeric complex responsible for the constitutive amiloride-sensitive inward Na⁺ current may lack an ASIC2 component As a result, inhibitors of the heteromeric complex, such as, but not limited to, syntaxin 1 and PKC, that are active in normal cells to inhibit the inward Na⁺ current are not effective.

### Example 6

### Effects of Amiloride and Analogs on Tumor Cell Proliferation and Invasion

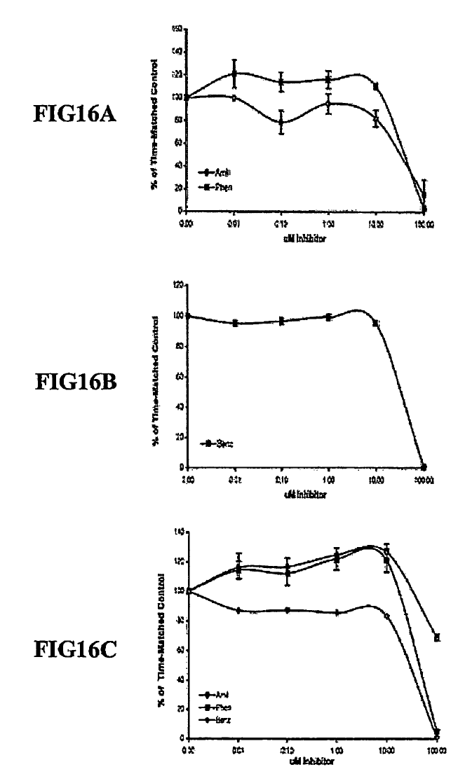

In order to examine the biological significance of the constitutive inward Na⁺ current, the ability of amiloride, phenamil, and benzamil to inhibit cell growth of three GBM cell lines using the MTT Cell Proliferation Assay was examined. If the Na⁺ conductance seen in high-grade glioma cells was required or linked to the high rate of cell growth, inhibition of the pathway should result in inhibition of cell growth and/or cell death. FIGS. 16A-C shows that the relative rate of proliferation for SKMG (FIG. 16A), U373 (FIG. 16B), and U251 (FIG. 16C) glioma cell lines is significantly inhibited at drug concentrations between 10-100 μM, the same concentration range at which the Na⁺ conductance is inhibited These results are complicated by the fact that high concentrations of amiloride can inhibit Na⁺/H⁺ exchange, a transport system also involved in glioma cell growth (72). However, benzamil is ineffective in inhibiting Na⁺/H⁺ exchange in glial cells (73), yet still can inhibit proliferation in this assay. Comparable results were seen in [³H]-thymidine incorporation experiments using U87-MG and SK-MG cells. Moreover, an amiloride analog that does not inhibit channel activity likewise did not affect [³H]-thymidine incorporation over the same concentration range. Therefore, inhibition of the inward Na⁺ current results in inhibition and/or stoppage of cell growth. These observations establish the importance of this pathway in tumor cell biology.

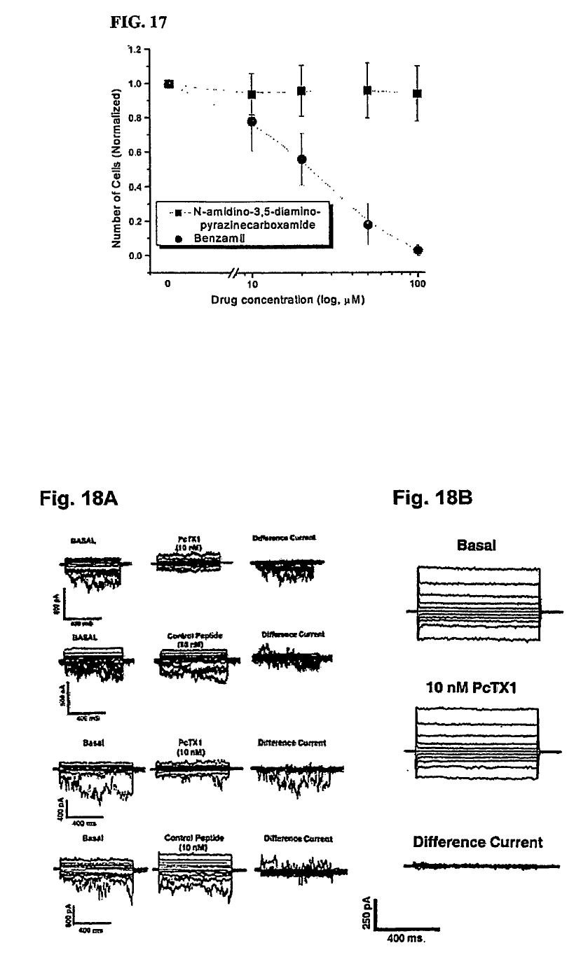

To begin to investigate the role of the inward Na⁺ conductance in the invasive behavior of tumor cells, a Transwell Migration Assay was used to assess cell chemotaxis and invasiveness (FIG. 17). D54-MG cells were plated on the upper side of a filter insert perforated with 5-8 μm holes, and induced to migrate through these pores toward the extracellular matrix protein vitronectin (coated on the underside of the filter). Benzamil, at 10, 20, 50, or 100 μM, was added to both chambers at time 0. After a 3 h migration time, cells were fixed, stained with crystal violet, and counted. BSA-coated filter inserts were used as negative controls. FIG. 17 shows that the migration of D54-MG glioma cells was inhibited by increasing concentrations of benzamil. Benzamil, in a concentration-dependent fashion, also inhibited Transwell migration of both U87-MG and SK-MG cells while the inactive amiloride analog (N-amidino-3,5-diamino-pyrazinecarboxamide) did not. The effective concentration range for inhibition of both proliferation and migration was the same as that necessary for inhibition of the inward Na⁺ current.

### Example 7

### Inward Na⁺ Currents are Sensitive to Psalmotoxin (PcTX1)

Psalmotoxin 1 (PcTX1) is a peptide isolated from the venom of the South American tarantula Psalmopoeus cambridgei. PcTX1 is a 40 amino acid peptide possessing 6 cysteine residues linked by three disulfide bridges. The amino acid sequence of PcTX1 is shown in SEQ ID NO: 1. PcTX1 has a limited homology with other spider toxins known in the art However, PcTX1 does share a conserved cysteine distribution found in both spider and cone snail peptide toxins (64).

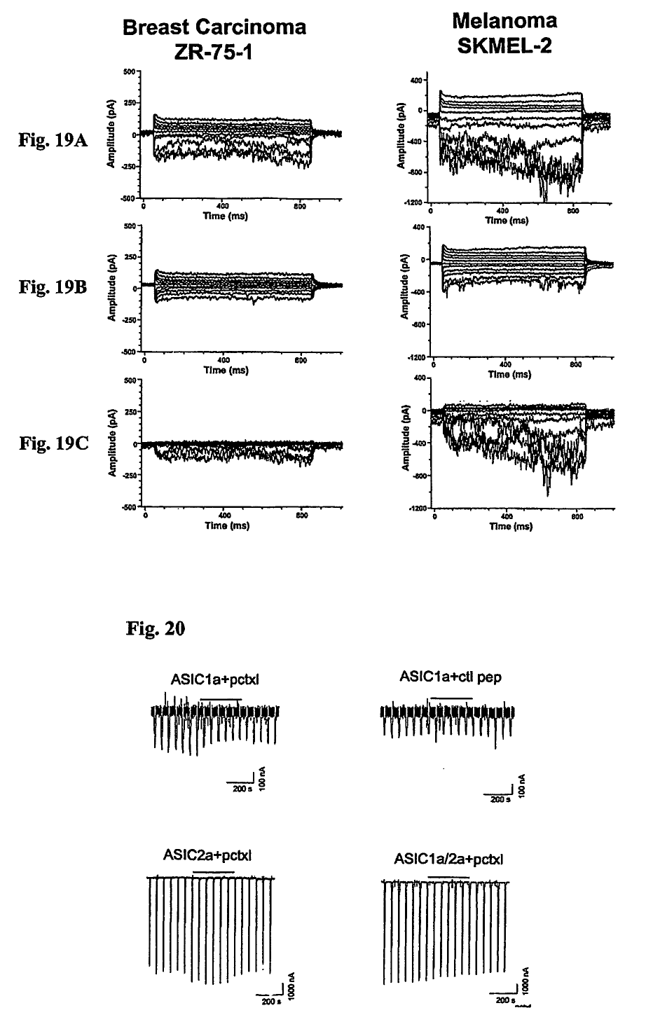

Constitutive amiloride-sensitive inward Na⁺ current in both a freshly resected GBM (upper two panels) and SK-MG cells (lower two panels) could be blocked by 10 nM synthetic PcTX1, but were left unaffected by a 40mer scrambled PcTX1 control peptide (the sequence of which is shown in SEQ ID NO: 3) having the same amino acid content as that shown in SEQ ID NO: 1 (FIG. 18A). PcTX1 similarly blocked inward basally activated currents in primary cultured GBM and in U87-MG. PcTX1 was without effect on whole-cell currents of normal human astrocytes (FIG. 18B). Therefore, PcTX1 is an inhibitor of the constitutive amiloride-sensitive inward Na⁺ current in high grade gliomas. In addition, constitutive amiloride-sensitive inward Na⁺ current of ZR-75-1 breast carcinoma cells and SKMEL-2 melanoma cells could be blocked by 10 nM synthetic PcTX1 (FIGS. 19A-C). FIG. 19A show representative whole-cell patch clamp recordings of ZR-75-1 and SKMEL-2 cells in the basal state. FIG. 19B shows the whole-cell patch clamp recordings in the presence of 100 uM PcTX1; FIG. 19C shows the PcTX1-sensitive difference current.

In oocytes PcTX1 blocked only inward currents mediated by ASIC1, and not those inward Na⁺ currents mediated by ASIC2 or the combination of ASIC1+ASIC2 (FIGS. 20A-D). Membrane potential was held at −60 mV, and the pHₒ was step decreased to 4.0 for 10 s, and then returned to 7.4 for 30 s before repeating the sequence. Oocytes were superfused with PcTX1 solution or control peptide solution (SEQ ID NO. 3) as indicated by the bars in the figures. Furthermore, PcTX1 blocked only inward currents mediated by ASIC1, not ASIC2 nor ASIC1+ASIC2 in planar lipid bilayers.

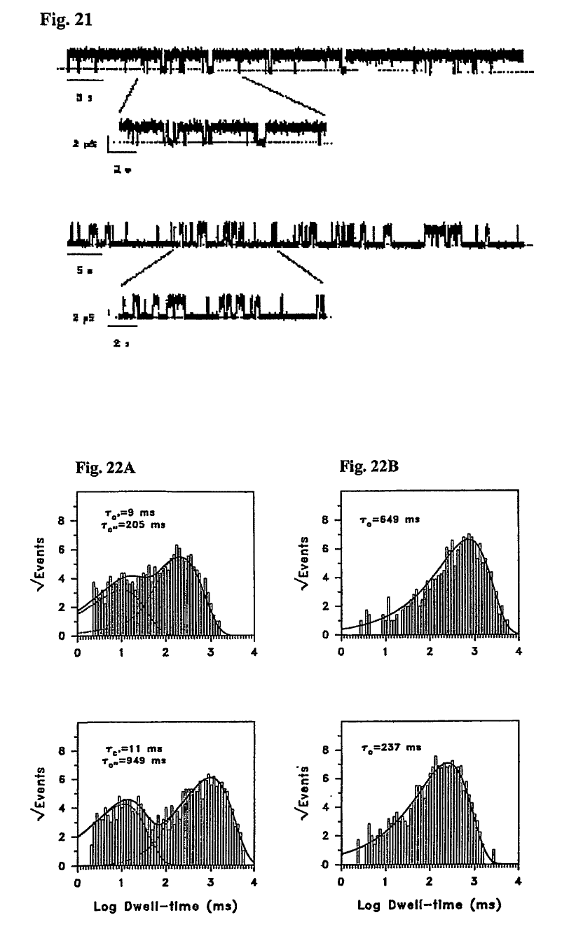

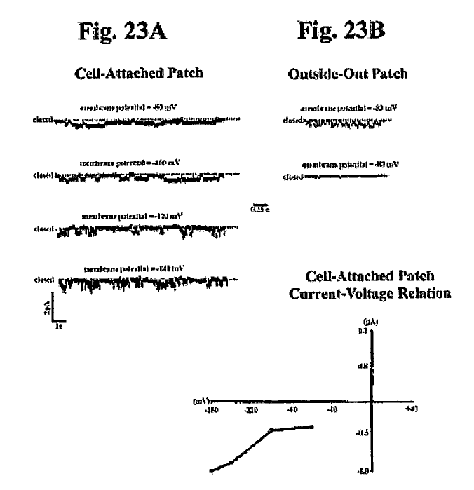

Moreover, analysis of long records of PcTX1 block of ASIC1 containing channels in planar lipid bilayers indicated that this toxin is a slow blocker of ASIC1 containing channel activity (FIG. 21). Single channel recordings of the ASIC1 containing channels reconstituted into planar lipid bilayers in the absence (FIG. 21 upper panel) and in the presence (FIG. 21, lower panel) of 10 nM PcTX1 were obtained. Control single channel were record for ASIC1 containing channels in bilayers bathed with symmetrical 100 mM NaCl, 10 mM MOPS, pH 6.2. Holding potential was +100 mV referred to the virtually grounded trans chamber. For illustration purposes, data shown were digitally filtered at 100 Hz using pCLAMP software (Axon Instruments) subsequent to acquisition of the analog signal filtered at 300 Hz with an 8-pole Bessel filter before acquisition at 1 ms per point. An expanded time scale is shown below each trace (FIG. 21).

The effect of PcTX1 on the kinetic properties of ASIC1 containing channels was also examined in planar lipid bilayers. FIG. 22A shows data obtained in the absence of PcTX1 and FIG. 22B illustrated data obtained in the presence of 10 nM PcTX1. Representative dwell-time histograms were constructed following the events analyses performed using pCLAMP software (Axon Instruments) on single channel recordings of 10 min in duration filtered at 300 Hz with an 8-pole Bessel filter before acquisition at 1 ms per point using pCLAMP software and hardware. The event detection thresholds were 50% in amplitude of transition between closed and open states, and 3 ms in duration. Closed and open time constants shown were determined by fitting the closed and open time histograms to the probability density function (Sigworth and Sine, 1987), and using the Simplex least square routine of pSTAT. Number of bins per decade in all histograms was 16. Numbers of events used for construction of the closed and open time histograms shown were: 811 and 812 in the absence of the PcTX1 (FIG. 22A) and 989 and 988 in the presence of 10 nM PcTX1 (FIG. 22B).

Single channel recording of ASIC containing channel activity in both cell-attached and outside-out patches from U-87MG cells are shown in FIGS. 23A and B, respectively. For cell-attached patches, the pipette solution contained RPMI 1640, matching the external bath solution used for all whole-cell clamped records. For outside-out patches the pipette solution contained (in mM) K-gluconate, 100; KCl, 30; NaCl, 10; HEPES, 20; EGTA, 0.5; ATP, 4; pH 7.2. Membrane potentials for cell-attached patches were determined as the applied potential plus the membrane potential of the cell that was measured in the whole-cell configuration as −60 mV using the pipette solution for outside-out patches. The membrane potential for outside-out patches was the equilibrium potential for sodium plus the applied potential. The average single channel conductance in the cell-attached configuration was 5.7±0.5 pS. This average conductance was calculated from each of the clamp potentials. This was compatible with the observed whole-cell currents. The kinetics of channel opening and closing were relatively slow (on the order of 0.1 to 1 s), consistent to what has been observed for ASIC-like channels in bilayers (FIG. 23A). Upon excision of the patch, outside-out recordings showed that channels could be completely inhibited with 100 μM amiloride (FIG. 23B).

### Example 8

### PcTX1 Blocks Migration, Regulatory Volume Increase and Cell Growth

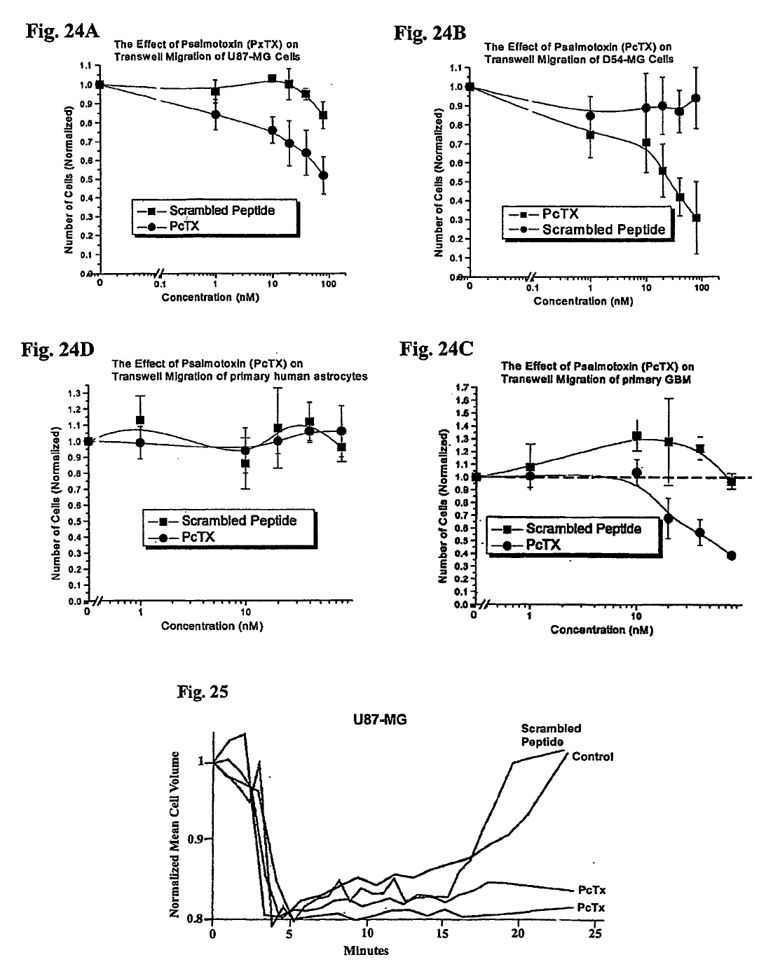

To further characterize the action of PcTX1, the effects of PcTX1 or control scrambled PcTX1 peptide (as described above) on migration and cell volume regulation were examined. FIGS. 24A-D show the results of Transwell migration assays of U87-MG cells (FIG. 24A), D54-MG cells (FIG. 24B), primary GBM cultures (FIG. 24C) and primary human astrocytes (FIG. 24D) in the presence of various concentrations of PcTX1 or control scrambled PcTX1 peptide. Approximately 10,000 cells were added to the upper side of a filter insert perforated with 5-8 μm holes, and induced to migrate through these pores toward the extracellular matrix protein vitronectin (coated on the underside of the filter). PcTX1 or control scrambled PcTX1 peptide was added to each compartment at the same time as the cells. BSA-coated filter inserts were used as negative controls. After 3 h, cells were fixed with 4% paraformaldehyde, and subsequently stained with crystal violet. Cells were counted on an inverted microscope, averaging five fields per Transwell chamber. Four-to-ten chambers were used under each condition. It can be seen that PcTX1 greatly (>90%) diminished the ability of U87-MG cells, D54-MG cells and primary GBM cultures to migrate through the filter, while the control scrambled PcTX1 peptide was without effect Furthermore, PcTX1 had no effect on the Transwell migration of primary human astrocytes.

80 nM PcTX1 effectively prevented U87-MG cells from recovering their volume after shrinkage (FIG. 25). U87-MG cells were mechanically dispersed, washed, and resuspended in PBS. At t=2-3 min, the osmolality of the bathing medium was increased to 450 mOsM/kg by the addition of NaCl from a 3M stock solution. The time course of volume recovery was continuously followed by Coulter counter analysis in the absence of peptide (control) or presence of 100 nM PcTX1 (SEQ ID NO: 1) or scrambled PcTX1 peptide (SEQ ID NO: 3).

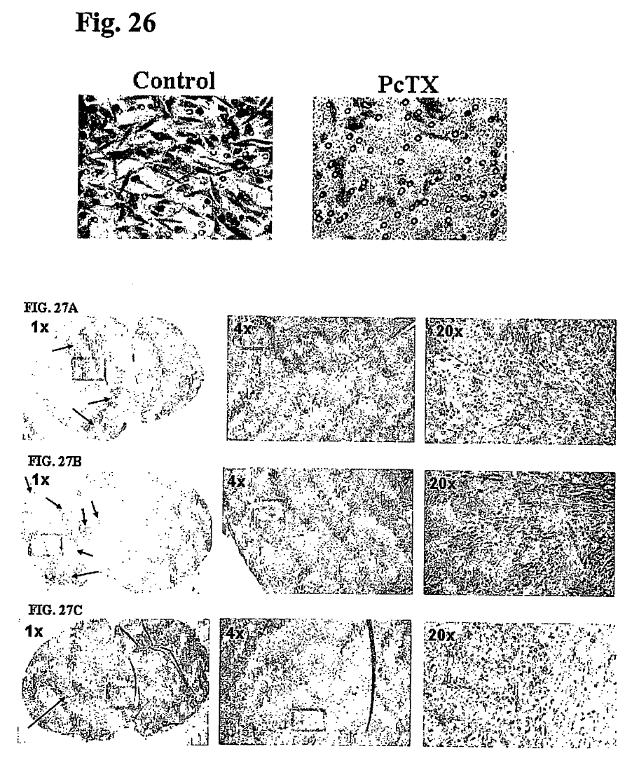

80 nM PcTX1 also inhibited the growth of U87-MG cells in culture. As can be seen in FIG. 26, the addition of 80 nM PcTX1 significantly inhibited the growth of U87-MG cells as compared to control cells where no PcTX1 was added.

### Example 9

### PcTX1 Decreases In-vivo Tumor Growth

Since PcTX1 inhibited the migration, volume recover and cell growth of glioma cells in an in vitro assay (see Example 8), PcTX1 was examined in a mouse xenograph model to see if administration of PcTX1 allowed better containment of intracranial tumors. In these studies, 10⁶ U251-MG cells were injected directly into the right hemisphere of thirty SCID mice (FIGS. 27A-C). The mice (3 groups of 10) were either treated by injection with saline (27A, upper panel), scrambled peptide (27B, middle panel) or PcTX1 (at 20× the in vitro inhibitory dose) (27C, lower panel) once a week for three weeks. On sacrifice of the animals, the brain of each mouse was sectioned and stained with hemotoxylin and eosin. Do to the nature of the study, no difference in survival between the three groups was noted. As can be seen in FIGS. 27A-C, the tumor margins were more clearly delineated in the PcTX1 -treated animals than in the saline-treated or scrambled peptide-treated controls. Moreover, PcTX1-treated animals showed only one tumor focus within the injected hemisphere, whereas the saline-treated or scrambled peptide-treated animals often showed 2 or 3 tumor foci within the injected hemisphere (FIGS. 27A-C).

These results suggest that the constitutive inward Na⁺ currents generated by the ion channels described herein play a role in tumor function and behavior. Furthermore, these results suggest that PcTX1 may be a candidate therapeutic agent, either alone or in combination with other drugs, for the treatment of tumors expressing the constitutive inward Na⁺ currents. In addition, the results demonstrate that PcTX1 may be used as a diagnostic probe to study and modulate the actions of the ion channels mediating the constitutive inward Na⁺ currents.

All references cited herein are incorporated by reference to the extent allowed. The references discussed herein are provided solely for their disclosure prior to the filing date of the present application. Nothing herein is to be construed as an admission that the inventors are not entitled to antedate such disclosure by virtue of prior disclosure.

### REFERENCES

1. Garty, H., and L. G. Palmer (1997). Epithelial Na⁺ Channels: function, structure, and regulation. Physiol. Rev. 77:359-396.
2. Benos, D. J., C. M. Fuller, V. Gh. Shlyonsky, B. K. Berdiev, and I. I. Ismailov (1997). Amiloride-sensitive Na⁺ channels: insights and outlooks. NIPS 12:55-61.
3. Benos, D. J., M. S. Awayda, I. I. Ismailov, and J. P. Johnson (1995). Structure and function of amiloride-sensitive Na⁺ channels. J. Membr. Biol. 143:1-18.
4. Price, M. P., P. M. Snyder, and M. J. Welsh (1996). Cloning and expression of a novel human brain Na⁺ channel. J. Biol. Chem. 271:7879-7882.
5. Garcia-Anoveros, J., B. Derfler, J. Neville-Golden, B. T. Hyman, and D. P. Corey (1997). BNaC1 and BNaC2 constitute a new family of human neuronal sodium channels related to degenerins and epithelial sodium channels. Proc. Natl. Acad. Sci. USA 94:1459-1464.
6. Biagini, G., K. Babinski, M. Avoli, M. Marcinkiewicz, and P. Seguela. (2001). Regional and subunit-specific downregulation of acid-sensing ion channels in the pilocarpine model of epilepsy. Neurobiol. Dis. 8:45-58.
7. Price, M. P., G. R Lewin, S. L. McIlwrath, C. Cheng, J. Xie, P. A. Heppenstall, C. L. Stucky, A. G. Mannsfeldt, T. J. Brennan, H. A. Drummond, J. Qiao, C. J. Benson, D. E. Tarr, R. F. Hrstka, B. Yang, R. A. Williamson, and M. J. Welsh. (2000). The mammalian sodium channel BNC1 is required for normal touch sensation. Nature 407:1007-1011
8. The Children's Brain Tumor Foundation Internet Site (www/childrensneuronet.org/med/index.html)
9. American Brain Tumor Association Internet Site (www.abta.org)
10. Schoenberg, B. S. (1983). Epidemiology of central nervous system tumor. In: Walker, M D, editor. Oncology of the Nervous System. Boston: Nijhoff; p. 1-30.
11. Levin, A. L., G. E. Shelin, and P. H. Gutin. (1989). Neoplasms of the central nervous system. In: Devita S., Hellman, S., Rosenberg, S. A., editors. Cancer: Principles and Practice of Oncology. 3^{rd} Ed., Philadelphia, Pa.: Lippincott; p. 1557-1611.
12. Kleihues, P., and H. Ohgaki. (1999). Primary and secondary glioblastomas: from concept to clinical diagnosis. Neuro-Oncol. 1:44-51.
13. Caffrey, J. M., A. M. Brown, and M. D. Schneider (1987). Mitogenes and oncogenes can block the induction of specific voltage-gated ion channels. Science 236:570-573.
14. Flamm, R. E., N. C. Birnberg, and L. K. Kaczmarek (1990). Transfection of activated ras into an excitable cell line (ATt-20) alters tetrodotoxin sensitivity of voltage-dependent sodium current Pflugers Arch. 416:120-125.
15. Grimes, J. A., S. P. Fraser, G. J. Stephens, J. E. G. Downing, M. D. Laniado, C. S. Foster, P. D. Abel, and M. B. A. Djamgoz (1995). Differential expression of voltage-activated Na⁺ currents in two prostatic tumour cell lines: contribution to invasiveness in vitro. FEBS Letters 369:290-294.
16. Hemmick, L. N, T. M. Perney, R. E. Flamm, L. K. Kaczmarek, and N. C. Birnberg (1992). Expression of the H-ras oncogene induces potassium conductance and neuro-specific potassium channel mRNAs in the AtT20 cell line. J. Neurosci. 12:2007-2014.
17. Huang, Y., and S. G. Rane (1993). Single channel study of a Ca²⁺-activated K⁺ current associated with ras-induced cell transformation. J. Physiol. 461:601-618.
18. Repp, H., H. Draheim, J. Ruland, G. Seidel, J. Beise, P. Presek, and F. Dreyer (1993). Profound differences in potassium current properties of normal and Rous sarcoma virus-transformed chicken embryo fibroblasts. Proc. Natl. Acad. Sci. USA 90:3403-3407.
19. Teulon, J., P. M. Ronco, M. Geniteau-Legendre, B. Baudouin, S. Estrade, R. Cassingena, and A. Vandewalle (1992). Transformation of renal tubule epithelial cells by simian virus-40 is associated with emergence of Ca²⁺-insensitive K⁺ channels and altered mitogenic sensitivity to K⁺ channel blockers. J. Cell Physiol. 151:113-125.
20. Chen, C. F., M. J. Corbley, R. M. Roberts, and P. Hess (1988). Voltage-sensitive calcium channels in normal and transformed 3T3 fibroblasts. Science 239:1024-1026.
21. Collin, C., A. G. Papageorge, D. R. Lowy, and D. L. Alkon (1990). Early enhancement of calcium currents by H-ras oncoproteins injected into Hermissenda neurons. Science 250:1743-1745.
22. Bading, H., D. D. Ginty, and M. E. Greenberg (1993). Regulation of gene expression in hippocampal neurons by distinct calcium signaling pathways. Science 260:181-186.
23. Mandel, G., S. S. Cooperman, R. A. Maue, R. H. Goodman, and P. Brehm (1988). Selective induction of brain type II Na⁺ channels by nerve growth factor. Proc. Natl. Acad. Sci. USA 85:924-928.
24. Gomez, M. P., G. Waloga, and E. Nasi (1993). Induction of voltage-dependent sodium channels by in vitro differentiation of human retinoblastoma cells. J. Neurophysiol. 70:1487-1496.
25. Asou, H. (1992). Monoclonal antibody that recognizes the carbohydrate portion of cell adhesion molecule L1 influences calcium current in cultured neurons. J. Cell Physiol. 153:313-320.
26. Silver, R. A. and S. R. Bolsover (1991). Expression of T-type calcium current precedes neurite extension in neuroblastoma cells. J. Physiol (Paris) 85:79-83.
27. Komuro, H. and P. Rakic (1992). Selective role of N-type calcium channels in neuronal migration. Science 257:806-809.
28. Becchetti, A., A. Arcangeli, M. R. Del Bene, M. Olivotto, and E. Wanke (1992). Response to fibronectin-integrin interaction in leukaemia cells: delayed enhancing of a K⁺ current Proc. Royal Soc. Lond. B 248:235-240.
29. Leonard, R. J., M. L. Garcia, R. S. Slaughter, and J. P. Reuben (1992). Selective blockers of voltage-gated K⁺ channels depolarize human T lymphocytes: mechanism of the antiproliferative effect of charybdotoxin. Proc. Natl. Acad. Sci. USA 89:10094-10098.
30. Nilius, B. and W. Wohlrab (1992). Potassium channels and regulation of proliferation of human melanoma cells. J. Physiol. 445:537-548.
31. Tsien, R. Y., T. Pozzan, and T. J. Rink (1982). T-cell mitogens cause early changes in cytoplasmic free Ca²⁺ and membrane potential in lymphocytes. Nature 295:68-71.
32. Ismailov, I. I, B. K. Berdiev, V. G. Shlyonsky, and D. J. Benos (1997). Mechanosensitivity of an epithelial Na⁺ channel in planar lipid bilayers: release from Ca²⁺ block. Biophys. J. 72(3):1182-1192.
33. Ji, H-L., C. M. Fuller, and D. J. Benos. (1998). Osmotic pressure regulates cloned rENaC expressed in Xenopus oocytes. Am. J. Physiol.:Cell Physiol. 275:C1182-C1190.
34. Waldmann, R. G. Champigny, N. Voilley, I. Lauritzen, and M. Lazdunski (1996). The mammalian degenerin MDEG, an amiloride-sensitive cation channel activated by mutations causing neurodegeneration in Caenorhabditis elegans. J. Biol. Chem. 271(18):10433-10436.
35. Cone, C. D., Jr. (1970). Variation of the transmembrane potential level as a basic mechanism of mitosis control. Oncology 24:438-470.
36. Cone, C. D., Jr. (1971). Unified theory on the basic mechanisms of normal mitotic control and oncogenesis. J. Theor. Biol. 30:151-181.
37. Platoshyn, O., V. A. Golovina, C. L. Bailey, A. Limsuwan, S. Krick, M. Juhaszova, J. E. Seiden, L. J. Rubin, and J. X.-J. Yuan. (2000). Sustained membrane depolarization and pulmonary artery smooth muscle cell proliferation. Am. J. Physiol.: Cell Physiol 279:C1540-C1549.
38. MacFarlane, S. N., H. Sontheimer. (2000). Changes in ion channel expression accompany cell cycle progression of spinal cord astrocytes. Glia. 30(1):39-48.
39. Waldmann, R., G. Champigny, N. Voilley, I. Lauritzen, and M. Lazdunski. (1996). The mammalian degenerin MDEG, an amiloride-sensitive cation channel activated by mutations causing neurodegeneration in Caenorhabditis elegans. J. Biol. Chem. 271:10433-10436.
40. Lingueglia, E., J. R. de Weille, F. Bassilana, C. Heurteaux, H. Sakai, R. Waldmann, and M. Lazdunski. (1997). A modulatory subunit of acid sensing ion channels in brain and dorsal root ganglion cells. J. Biol. Chem. 272:29778-29783.
41. Champigny, G., N. Voilley, R. Waldmann, and M. Lazdunski. (1998). Mutations causing neurodegeneration in Caenorhabditis elegans drastically alter the pH sensitivity and inactivation of the mammalian H⁺-gated Na⁺ channel MDEG1. J. Biol. Chem. 273:15418-15422.
42. Sakai, H., E. Lingueglia, G. Champigny, M.-G. Mattei, and M. Lazdunski. (1999). Cloning and functional expression of a novel degenerin-like Na⁺ channel gene in mammals. J. Physiol. 519:323-333.
43. Bassilana, F., G. Champigny, R. Waldmann, J. R. de Weille, C. Heurteaux, and J. Lazdunski. (1997). The acid-sensitive ionic channel subunit ASIC and the mammalian degenerin MDEG form a heteromultimeric H⁺-gated Na⁺ channel with novel properties. J. Biol. Chem. 272:28819-28822.
44. Babinski, K., S. Catarsi, G. Biagini, and P. Seguela. (2000). Mammalian ASIC2a and ASIC3 subunits co-assembled into heteromeric proton-gated channels sensitive to Gd³⁺. J. Biol. Chem. 275:28519-28525.
45. Chesler, M., and K. Kaila. (1992). Modulation of pH by neuronal activity. Trends Neurosci. 15:396-402.
46. Babinski, K., K.-T. Le, and P. Seguela. (1999). Molecular cloning and regional distribution of a human proton receptor subunit with biphasic functional properties. J. Neurochem. 72:51-57.
47. Paschen, W., B. Djuricic, G. Mies, R. Schmidt-Kastner, and F. Linn. (1987). Lactate and pH in the brain: association and dissociation in different pathophysiological states. J. Neurochem. 48:154-159.
48. Laxer, K. D. (2000). Phosphorous MRS: pH, ATP, PCr. Adv. Neurol. 83:273-277.
49. Biagini, G., K. Babinski, M. Avoli, M. Macinkiewicz, and P. Seguela. (2001). Regional and subunit-specific downregulation of acid-sensing ion channels in the pilocarpine model of epilepsy. Neurobiol. Dis. 8:5-48.
50. Adams, C. M., P. M. Snyder, M. P. Price, and M. J. Welsh. (1998). Protons activate brain Na⁺ channel 1 by inducing a conformational change that expose a residue associated with neurodegeneration. J. Biol. Chem. 273:30204-30207.
51. Rothman, J. E. (1994). Mechanisms of intracellular protein transport Nature 372:55-63.
52. Fasshauer, D., R. B. Sutton, A. T. Brunger, and R. Jahn. (1998). Conserved structural features of the synaptic fusion complex: SNARE proteins reclassified as Q- and R-SNARES. Proc. Acad. Sci. 95:15791-15786.
53. Naren, A. P., D. J. Nelson, W. Xe, B. Jovov, B. Peysner, M. K. Bennett, D. J. Benos, M. W. Quick and K. L. Kirk. (1997). Regulation of CFTR chloride channels by syntaxin and Munc 18 isoforms. Nature 390:302-305.
54. Naren, A. P., A. Di, E. Cormet-Boyaka, P. N. Boyaka, J. R. McGhee, K. Akagawa, T. Fujiwara, U. Thome, J. F. Engelhardt, D. J. Nelson, and K. L. Kirk. (2000). Syntaxin 1A is expressed in airway epithelial cells where it modulates CFTR C1⁻ currents. J. Clin. Invest. 105:377-386.
55. Peters, K, W., J. Qi, S. C. Watkins, and R. A. Frizzell. (1999). Syntaxin 1A inhibits regulated CFTR trafficking in Xenopus oocytes. Am. J. Physiol. 277:C174-C180.
56. Saxena, S., M. W. Quick, A. Tousson, Y. Oh, and D. G. Warnock. (1999). Interaction of syntaxins with the amiloride-sensitive epithelial sodium channel. J. Biol. Chem. 274:20812-20817.
57. Qi, J., C. Peters, K, W. Peters, C. Liu, J.-M. Wang, R. S. Edinger, J. P. Johnson, S. C. Watkins, and R. A. Frizzell. (1999) Regulation of the amiloride-sensitive epithelial sodium channels by syntaxin 1A. J. Biol. Chem. 274:30345-30348.
58. Naren, A. P., M. W. Quick, J. F. Collawn, D. J. Nelson and K. L. Kirk. (1998). Syntaxin 1A inhibits CFTR chloride channels by means of domain-specific protein-protein interactions. Proc. Natl. Acad. Sci. 95:10972-10977.
59. Bezprozvanny, I., R. H. Scheller, and R. W. Tsien. (1995). Functional impact of syntaxin on gating of N-type and Q-type calcium channels. Nature 378:623-626.
60. Rettig, J., C. Heinemann, U. Ashery, Z. H. Sheng, C. T. Yokoyama, W. A. Catterall, et al. (1997) Alterations of Ca²⁺ dependence of neurotransmitter release by disruption of Ca²⁺ channel/syntaxin interaction. J. Neuroscience 17:6647-6656.
61. Wiser, O., A. Trus, E. Renstrom, S. Barq, P. Rorsman, and D. Atlas. (1999). The voltage-sensitive Lc-type Ca²⁺ channel is functionally coupled to the exocytotic machinery. Proc. Natl. Acad. Sci. 96:248-253.
62. Zhong, H., C. T. Yokoyama. T. Scheuer and W. A. Catterall. (1999). Reciprocal regulation of P/Q-type Ca channels by SNAP-25, syntaxin and synaptotagmin. Nature Neurosci. 2:939-941.
63. Sheng, Z. H., J. Retting, J. Takahashi, and W. A. Catterall. (1994). Identification of a syntaxin-binding site on N-type calcium channels. Neuron 13:1303-1313.
64. Escoubas, P., J. R. DeWeille, A. Lecoq, S. Diochot, R. Waldmann, G. Champigny, D. Moinier, A. Menez, and M. Lazdunski. (2000). Isolation of a tarantula toxin specific for a class of proton-gated Na⁺ channels. J. Biol. Chem. 275:25116-25121.
65. Chu, X. P. J. Miesch, M. Johnson, L. Root, X. M. Zhu, D. Chen, R. P. Simon, Z. G. Xiong. (2002). Proton-gated channels in PC12 cells. J. Neurophysiol. 87:2555-2561.
66. Ashcroft, F. M. (2000). Ion channels and disease. Academic Press, San Diego, pp 481.
67. Awayda, M. A., I. I. Ismailov, B. K. Berdiev, C. M. Fuller, and D. J. Benos (1996). Protein kinase regulation of a cloned epithelial Na⁺ channel. J. Gen. Physiol. 108:49-65.
68. Ji H L, M. L. Chalfant, B. Jovov, J. P. Lockhart, S. B. Parker, C. M. Fuller, B. A. Stanton, and D. J. Benos (2000). The cytosolic termini of the β- and γ-ENaC subunits are involved in the functional interactions between cystic fibrosis transmembrane conductance regulator and epithelial sodium channel. J. Biol. Chem. 275:27947-27956.
69. Jovov, B., A. Tousson, H.-L. Ji, D. Keeton, V. Shlyonsky, P. J. Ripoll, C. M. Fuller and D. J. Benos (1999). Regulation of epithelial Na⁺ channels by actin in planar lipid bilayers and in the Xenopus oocyte expression system. J. Biol. Chem. 274:37845-37854.
70. Tavernarakis, N., J. K. Everett, N. C. Kyrpides, M. Driscoll (2001). Structural and functional features of the intracellular amino terminus of Deg/ENaC ion channels. Curr. Biol. 11:R205-208.
71. Chung, S., T. L. Gumienny, M. O. Hengartner, and M. Driscoll. A common set of engulfment genes mediates removal of both apoptotic and necrotic cell corpuses in C. elegans. Nat. Cell Biol. 2:931-917.
72. McLean, L. A., J. Roscoe, N. K Jorgensen, F. A. Gorin, and P. M. Cala. (2000). Malignant gliomas display altered pH regulation by NHE1 compared with nontransformed astrocytes. Am. J. Physiol.: Cell Physiol. 278:C676-C688.
73. Benos, D. J. and V. S. Sapirstein (1983). Characteristics of an amiloride-sensitive sodium entry pathway in cultured rodent glial and neuroblastoma cells. J. Cell Physiol. 116:213-220.
74. Copeland, S. J., Berdiev, B. K, Lockhart, J., Parker, S., Fuller, C. M., and Benos, D. J. (2001) Regions in the carboxy-terminal of αbENaC involved in gating and actin binding. Am. J. Physiol. Cell Physiol. 281:C231-C240.
75. Berdiev, B. K., R. Latorre, D. J. Benos, and I. I. Ismailov (2001). Actin modifies Ca²⁺ block of epithelial Na⁺ channels in planar lipid bilayers. Biophys. J. 80:2176-2186.
76. Miller, C., ed. (1986). Ion Channel Reconstitution. Plenum Press, New York, pp. 577.
77. Berdiev, B. K., R. Latorre, D. J. Benos, I. I. Ismailov. (2001). Actin modifies Ca²⁺ block of epithelial Na⁺ channels in planar lipid bilayers. Biophys. J. 80:2176-2186.
78. Corey, D. R., and J. M. Abrams. (2001). Morpholino antisense oligonucleotides: tools for investigating vertebrate development Genome Biol. 2:1015.1-1015.3
79. Heasman, J. (2002). Morpholino oligos: making sense of antisense? Dev. Biol. 243:209-214.
80. Ekker, S. C. (2000). Morphants: a new systematic vertebrate functional genomics approach. Yeast 17:302-306.
81. Kaneko, K., K. Satoh, A. Masamune, A. Satoh, and T. Shimosegawa. (2002) Expression of ROCK-1 in human pancreatic cancer: it's down-regulation by morpholino oligo antisense can reduce the migration of pancreatic cancer cells in vitro. Pancreas, 24:251-257.
82. Swertfeger, D. K., G. Bu, and D. Y. Hui. (2002). Low densitiy lipoprotein receptor-related protein mediates apolipoprotein E inhibition of smooth muscle cell migration. J. Biol. Chem. 277:4141-4146.
83. Qin, G., M. Taylor, Y. Y. Ning, P. Iversen and L. Kobzik. (2000). In vivo evaluation of a morpholino antisense oligomer directed against tumor necrosis factor alpha. Antisense Nucl. Acid Drug Dev. 10: 11-16.
84. Coonrod, S. A., L. C. Bolling, P. W. Wright, P. E. Visconti, and J. C. Herr. (2001). A morpholino phenocopy of the mouse MOS mutation. Genesis, 30:198-200.
85. Summerton, J., D. Stein, S. B. Huang, P. Matthews, D. Weller and M. Partridge. (1997). Morpholino and phosphorothioate antisense oligomers compared in cell-free and in-cell systems. Antisense Nucl. Acid Drug Dev. 7:63-70.
86. Partridge, M., A. Vincent, P. Matthews, J. Puma, D. Stein, and J. Summerton. (1996). A simple method for delivering morpholino antisense oligos into the cytoplasm of cells. Antisense Nucl. Acid Drug Dev. 6:169-175.
87. Morcos, P. A. (2001) Achieving efficient delivery of morpholino oligos in cultured cells. Genesis, 30:94-102.
88. Brantl, S. Antisense-RNA regulation and RNA interference. (2002). Biochim. Biophys. Acta 1575: 15-25.
89. Hannon, G. J. (2002). RNA interference. Nature 418:244-251.
90. Nishikura, K. (2001) A short primer on RNAi: RNA-directed RNA polymerase acts as a key catalyst. Cell, 107: 415-418.
91. Fire, A., S. Xu, M. Montgomery, S. Kostas, S. Driver, and C. Mello. (1998). Potent and specific genetic interference by double stranded RNA in Caenorhabditis elegans. Nature, 391:806-811.
92. Tuschl, T, P. D. Zamore, R. Lehmen, D. P. Bartel, and P. A. Sharp. (1999). Targeted mRNA degradation by double-stranded RNA in vitro. Genes Dev. 13:3191-3197.
93. Bernstein, E., A. Caudy, S. Hammond, and G. Hannon. (2001). Role for a bidentate ribonuclease in the initiation step of RNA interference. Nature, 409:363-366.
94. Nykanen, A., B. Haley, and P. D. Zamore. (2001). ATP requirements and small interfering RNA structure in the RNA interference pathway. Cell 107: 309-321.
95. Sijen, T., J. Fleenor, F. Simmer, K. L. Thijssen, S. Parrish, L. Timmons, R. H. A. Plasterk, and A. Fire. (2001). On the role of RNA amplification in dsRNA-triggered gene silencing. Cell 107:465-476.
96. Lipardi, C., Q. Wei, and B. M. Paterson. (2001). RNAi as random degradative PCR: siRNA primers convert mRNA into dsRNAs that are degraded to generate new siRNAs. Cell 107:297-307.
97. Elbashir, S. M., J. Harborth, W. Lendeckel, A. Yalcin, K. Weber, and T. Tuschl. (2001). Duplexes of 21-nucleotide RNAs mediate RNA interference in cultured mammalian cells. Nature 411:494-498.
98. Harborth, J., S. M. Elbashir, K. Bechert, T. Tuschl, and K. Weber. (2001) Identification of essential genes in cultured mammalian cells using small interfering RNAs. J. Cell Sci. 114:4557-4565.
99. Krichevsky, A. M. and K. S. Kosik. (2002). RNAi functions in cultured mammalian neurons. Proc. Natl. Acad. Sci. USA 99:11926-11929.
100. Yu, J.-Y., S. L. DeRuiter, and D. L. Turner. (2002) RNA interference by expression of short-interfering RNAs and hairpin RNAs in mammalian cells. Proc. Natl. Acad. Sci. USA 99:6047-6052.
101. Paddison, P. J., A. A. Caudy and G. J. Hannon (2002). Stable expression of gene expression by RNAi in mammalian cells. Proc. Natl. Acad. Sci. USA 99:1443-1448.
102. Tuschl, T., S. Elbashir, J. Harborth, and K. Weber. (2002). The siRNA user guide. www.mpibpc.gwdg.de/abteilungen/100/105/sirna_u.html.
103. Donze, O., and D. Picard. (2002). RNA interference in mammalian cells using siRNAs synthesized with T7 RNA polymerase. Nucl. Acids Res. 30: Epub. ahead of print.
104. Byrom, M., V. Pallotta, D. Brown and L. Ford. Visualizing siRNA in mammalian cells. (2002). Ambion Tech Notes 9. www.ambion.com/techlib/tn/93/935.html
105. Brown, D., R Jarvis, V. Pallotta, M. Byrom, and L. Ford. (2002) RNA interference in mammalian cell culture: design, execution and analysis of the siRNA effect Ambion Tech Notes 9: www.ambion.com/techlib/tn/91/912.html.
106. DuVall, M. D., S. Zhu, C. M. Fuller, and S. Matalon. (1998) Peroxynitrite inhibits amiloride-sensitive Na⁺ currents in Xenopus oocytes expressing αβγrENaC. Am. J. Physiol. Cell Physiol. 274:C1417-C1423.
107. Kisielow, M., S. Kleiner, M. Nagasawa, A. Faisal, and Y. Nagamine. (2002). Isoform-specific knockdown and expression of adaptor protein ShcA using small interfering RNA. Biochem. J. 363: 1-5.
108. Matthews, C. J., J. A. Tabcharani, X. B. Chang, T. J. Jensen, J. R. Riordan, and J. W. Hanrahan (1998). Dibasic protein kinase A sites regulate bursting rate and nucleotide sensitivity of the cystic fibrosis transmembrane conductance regulator chloride channel. J. Physiol. 508:365-377.
109. Naren, A. P., E. Cormet-Boyaka, J. Fu, M. Villain, J. E. Blalock, M. W. Quick, and K. L. Kirk (1999). CFTR chloride channel regulation by an interdomain interaction. Science 286:544-548.
110. Watkins, B. A., H. H. Dorn, W. B. Kelly, R. C. Armstrong, B. J. Potts, F. Michaels, C. V. Kufta, M. Dubois-Dalcq (1990). Specific tropism of HIV-1 for microglial cells in primary human brain cultures. Science 249:549-553.
111. DeBin, J. A. and G. R. Strichartz. (1991). Chloride channel inhibition by the venom of the scorpion Leiurus quinquestriatus. Toxicon 29:1403-1408.
112. Moran, O. (2001). Molecular simulation of the interaction of κ-conotoxin-PVIIA with the Shaker potassium channel pore. Eur. Biophys. J. 30:528-536.
113. Suchyna, T. M., J. H. Johnson, K. Hamer, J. F. Leykam, D. A. Gage, H. F. Clemo, C. M. Baumgarten, and F. Sachs. (2000) Identification of a peptide toxin from Grammostola spatulata spider venom that blocks cation-selective stretch-activated channels. J. Gen. Physiol. 115:583-598.
114. Cummins, T. R., F. Aglieco, and S. D. Dib-Hajj. (2002) Critical molecular determinants of voltage-gated sodium channel sensitivity to μ-conotoxins GIIIA/B. Molec. Pharmacol. 61:1192-1201.
115. McDonough, S. I., L. M. Boland, I. M. Mintz, and B. P. Bean. (2002). Interactions among toxins that inhibit N-type and P-type calcium channels. J. Gen. Physiol. 119:313-328.
116. Pardo-Lopez, L., J. Garcia-Valdes, G. B. Gurrola, G. A. Robertson, L. D. Possani. (2002). Mapping the receptor site for ergtoxin, a specific blocker of ERG channels. FEBS Letters 510:45-49.
117. Pardo-Lopez, L., M. Zhang, J. Liu, M. Jiang, L. D. Possani, and G.-N Tseng. (2002). Mapping the binding site of a human ether-a-go-go-related gene-specific peptide toxin (ErgTx) to the channel's outer vestibule. J. Biol. Chem. 277:16403-16411.
118. Gough, J. D., R. H. Williams, Jr., A. E. Donofrio, and W. J. Lees. (2002). Folding disulfide-containing proteins faster with an aromatic thiol. J. Am. Chem. Soc. 124:3885-3892.
119. Jackson, P. J., J. C. McNulty, Y.-K. Yang, D. A. Thompson, B. Chai, R. Gantz, G. S. Barsh, and G. L. Millhauser. (2002). Design, pharmacology, and NMR structure of a minimized cystine knot with agouti-related protein activity. Biochemistry 41:7565-7572.
120. Xu, Y., M. J. Jablonsky, P. L. Jackson, W. Braun, and N. R. Krishna. (2001). Automatic assignment of NOESY cross peaks and determination of the protein structure of a new world scorpion neurotoxin using NOAH/DIAMOD. J. Magn. Res. 148:35-46.
121. Jablonsky, M. J., P. L. Jackson, J. O. Trent, D. D. Watts, and N. R. Krishna. (1999). Solution structure of a β-neurotoxin from the new world scorpion Centruroides sculpturatus ewing. Biochem. Biophys. Res. Comm. 254:406-412.
122. Villain, M., P. L. Jackson, M. K. Manion, W.-J. Dong. Z. Su, G. Fassina, T. M. Johnson, T. T. Sakai, N. R. Krishna, and J. E. Blalock. (2000). De novo design of peptides targeted to the EF hand of calmodulin. J. Biol. Chem. 274:2676-2685.
123. Jablonsky, M. J., P. L. Jackson, and N. R. Krishna. (2002). Solution structure of an insect-specific neurotoxin from the new world scorpion Centruroides sculpturatus ewing. Biochemistry. 40:8273-8282.
124. Sambrook, I., E. F. Fritsch, and T. Maniatis. (1989). Molecular cloning: a laboratory manual. Cold Springs Harbor: Cold Springs Harbor Laboratory Press.
125. Selden, R. F. (1989). Transfection using DEAE-Dextran. In: Current Protocols in Molecular Biology, Ausubel, F. M. R. Burt, R. E. Kingston, D. d. Moore, J. G. Seidman, J. A. Smith, and K. Struhl, eds. Unit 9.2, Greene Publishing: New York.
126. Palmer, L. G., and G. Frindt. (1996). Gating of Na channels in the rat cortical collecting tubule: effects of voltage and membrane stretch. J. Gen. Physiol. 107:35-45.
127. Hooper, J. D., Clements, J. A., Quigley, J. P., and Antalis, T. M. (2001) J Biol Chem 276, 857-860.
128. Dickson, R. B., Johnson, M. D., el-Ashry, D., Shi, Y. E., Bano, M., Zugmaier, G., Ziff, B., Lippman, M. E., and Chrysogelos, S. (1993) Adv Exp Med Biol 330, 119-141.
129. Lin, C. Y., Wang, J. K, Torri, J., Dou, L., Sang, Q. A., and Dickson, R. B. (1997) J Biol Chem 272, 9147-9152.
130. Lin, C. Y., Anders, J., Johnson, M., Sang, Q. A., and Dickson, R. B. (1999) J Biol Chem 274, 18231-18236.
131. Oberst, M., Anders, J., Xie, B., Singh, B., Ossandon, M., Johnson, M., Dickson, R. B., and Lin, C. Y. (2001) Am J Pathol 158, 1301-1311.
132. Lin, C. Y., Anders, J., Johnson, M., and Dickson, R. B. (1999) J Biol Chem 274, 18237-18242.
133. Takeuchi, T., Shuman, M. A., and Craik, C. S. (1999) Proc Natl Acad Sci U S A 96, 11054-11061.

134. Ihara, S., Miyoshi, E., Ko, J. H., Murata, K, Nakahara, S., Honke, K., Dickson, R. B., Lin, C. Y., and Taniguchi, N. (2002) J Biol Chem 277, 16960-16967.

TABLE 1
Terminology for Acid Sensing Ion Channels

| Name | Psuedonymn | Distribution |
|---|---|---|
| ASIC1a | ASIC; BNaC2; BNC2; ACCN2 | Sensory neurons of DR and trigenminal gangli [symbol] |
| ASIC1b | ASICb; BNaC2 (alt. spliced) | Sensory neurons |
| ASIC2a | BNaC; MDEG; BNC1; ACCN1 | Widespread in nervous system |
| ASIC2b | MDEG2 | Sensory neurons |
| ASIC3(a, b, c) | TNAC1; SLNAC1; ACCN3; (DRASIC) | Sensory ganglia, brain, testis, lung |
| ASIC4 | | Pituitary, brain, spinal cord, inner ear |
| hINac | BLINaC | Small intestine |

1-38. (canceled)

39. A method of treating a tumor in a subject in need of such treatment, said tumor characterized by an expression of a Na⁺ channel mediating a constitutive inward Na⁺ current, said method comprising administering an effective amount of a pharmaceutical composition comprising an agent that binds to a component of said Na⁺ channel.

40. The method of claim 39 where said agent inhibits the activity of said Na⁺ channel.

41. The method of claim 39 where said agent is a polypeptide.

42. The method of claim 41 where said polypeptide is selected from a group consisting of: PcTX1, and a variant of PcTX1.

43. The method of claim 42 where said polypeptide is linked to a cytotoxic agent.

44. The method of claim 43 wherein the cytotoxic agent is selected from the group consisting of a radiolabel, gelonin, ricin, saponin, pseudomonas exotoxin, pokeweed antiviral protein, diphtheria toxin and complement proteins.

45. The method of claim 44 wherein the radiolabel is selected from the group consisting of ¹³¹I and ¹²⁵I.

46. The method of claim 42 where said PcTX1 has an amino acid sequence encoded by SEQ ID NO. 1 or SEQ ID NO. 2.

47. The method of claim 39 wherein the tumor is derived from a glial cell.

48. The method of claim 47 wherein the tumor is a glioma.

49. The method of claim 48 wherein the glioma is selected from the group consisting of astrocytoma, glioblastoma and medulloblastoma.

50. The method of claim 39 where the tumor is derived from an epithelial cell.

51. The method of claim 50 where the tumor is a breast carcinoma.

52. The method of claim 39 where the tumor is derived from a melanocyte.

53. The method of claim 52 where the tumor is a melanoma.

54. The method of claim 39 where said Na⁺ channel has an ASIC1 component.

55. The method of claim 39 where said Na⁺ channel lacks a functional ASIC2 component.

56. The method of claim 39 where said agent can be administered by routes selected from the group consisting of intravenous, intramuscular, intracranial, intraperitoneal, transmucosal, topical and oral routes.

57. The method of claim 39 where the dose of said agent is selected from the range consisting of 0.1 to 100 pg/kg.

58. The method of claim 39 where the subject is a human.

59. A method of treating a tumor in a subject in need of such treatment, said tumor characterized by an expression of a Na⁺ channel mediating a constitutive inward Na⁺ current, said method comprising administering an effective amount of a pharmaceutical composition comprising an agent fused to a tag protein, said agent binding to a component of said Na⁺ channel.

60. The method of claim 59 further comprising the step of administering a compound which binds to the tag protein.

61. The method of claim 60 wherein the compound is an antibody.

62. The method of claim 61 wherein the antibody is monoclonal.

63. The method of claim 61 wherein the antibody binds to the tag protein.

64. The method of claim 63 where the tag protein is glutathione-S-transferase.

65. The method of claim 60 wherein the compound is linked to a cytotoxic agent.

66. The method of claim 65 wherein the cytotoxic agent is selected from the group consisting of a radiolabel, gelonin, ricin, saponin, pseudomonas exotoxin, pokeweed antiviral protein, diphtheria toxin and complement proteins

67. The method of claim 66 wherein the radiolabel is selected from the group consisting of ¹³¹I and ¹²⁵I.

68. The method of claim 59 where said compound inhibits the activity of said Na⁺ channel.

69. The method of claim 59 where said compound is a polypeptide.

70. The method of claim 69 where said polypeptide is selected from a group consisting of: PcTX1 and a variant of PcTX1.

71. The method of claim 59 wherein the tumor is derived from a glial cell.

72. The method of claim 71 wherein the tumor is a glioma.

73. The method of claim 72 wherein the glioma is selected from the group consisting of astrocytoma, glioblastoma and medulloblastoma.

74. The method of claim 59 where the tumor is derived from an epithelial cell.

75. The method of claim 74 where the tumor is a breast carcinoma.

76. The method of claim 59 where the tumor is derived from a melanocyte.

77. The method of claim 76 where the tumor is a melanoma.

78. The method of claim 59 where said compound can be administered by routes selected from the group consisting of intravenous, intramuscular, intracranial, intraperitoneal, transmucosal, topical and oral routes.

79. The method of claim 59 where the dose of said compound is selected from the range consisting of 0.1 to 100 pg/kg.

80. The method of claim 59 where the subject is a human.

81. A method of treating a glioma in a subject in need of such treatment said method comprising administering an effective amount of a pharmaceutical composition comprising PcTX1 or a variant of PcTX1 linked to a cytotoxic agent.

82. The method of claim 81 where said glioma is characterized by an expression of a Na⁺ channel mediating a constitutive inward Na⁺ current.

83. The method of claim 82 wherein the glioma is selected from the group consisting of astrocytoma, glioblastoma and medulloblastoma.

84. The method of claim 81 wherein the cytotoxic agent is selected from the group consisting of a radiolabel, gelonin, ricin, saponin, pseudomonas exotoxin, pokeweed antiviral protein, diphtheria toxin and complement proteins.

85. The method of claim 84 wherein the radiolabel is selected from the group consisting of ¹³¹I and ¹²⁵I.).

86. A method of treating a breast carcinoma in a subject in need of such treatment said method comprising administering an effective amount of a pharmaceutical composition comprising PcTX1 or a variant of PcTX1 linked to a cytotoxic agent.

87. The method of claim 86 where said breast carcinoma is characterized by an expression of a Na⁺ channel mediating a constitutive inward Na⁺ current.

88. The method of claim 87 wherein the cytotoxic agent is selected from the group consisting of a radiolabel, gelonin, ricin, saponin, pseudomonas exotoxin, pokeweed antiviral protein, diphtheria toxin and complement proteins.

89. The method of claim 88 wherein the radiolabel is selected from the group consisting of ¹³¹I and ¹²⁵I.).

90. A method of treating a melanoma in a subject in need of such treatment said method comprising administering an effective amount of a pharmaceutical composition comprising PcTX1 or a variant of PcTX1 linked to a cytotoxic agent.

91. The method of claim 90 where said melanoma is characterized by an expression of a Na⁺ channel mediating a constitutive inward Na⁺ current.

92. The method of claim 91 wherein the cytotoxic agent is selected from the group consisting of a radiolabel, gelonin, ricin, saponin, pseudomonas exotoxin, pokeweed antiviral protein, diphtheria toxin and complement proteins.

93. The method of claim 92 wherein the radiolabel is selected from the group consisting of ¹³¹I and ¹²⁵I.).

94. A method of diagnosis to identify individuals with tumors characterized by a constitutive inward Na⁺ current, said method comprising administering a diagnostically effective amount of a PcTX1 or a variant of PcTX1 linked to a diagnostic agent to a subject in need of said diagnosis.

95. The method of claim 94 wherein the diagnostic agent is selected from the group consisting of a radiolabel and a fluorescent label.

96. The method of claim 95 wherein the radiolabel is selected from the group consisting of ¹³¹I and ¹²⁵I.

97. The method of claim 94 wherein the tumor is derived from a glial cell.

98. The method of claim 97 wherein the tumor is a glioma.

99. The method of claim 98 wherein the glioma is selected from the group consisting of astrocytoma, glioblastoma and medulloblastoma.

100. The method of claim 94 where the tumor is derived from an epithelial cell.

101. The method of claim 100 where the tumor is a breast carcinoma.

102. The method of claim 94 where the tumor is derived from a melanocyte.

103. The method of claim 102 where the tumor is a melanoma.

104. The method of claim 94 where said Na⁺ current is mediated by a Na⁺ channel having an ASIC1 component.

105. The method of claim 94 where said Na⁺ current is mediated by a Na⁺ channel lacking a functional ASIC2 component.

106. The method of claim 94 where the subject is a human.

107. A method of identifying agents that bind to a Na⁺ channel mediating a constitutive inward Na⁺ current, said method comprising the steps of:
a. providing a system comprising said Na⁺ channel comprising at least one ASIC component mediating said constitutive inward Na⁺ current in a functional state;
b. contacting said system with a test compound; and
c. measuring the binding of said test compound to said Na⁺ channel.

108. The method of claim 107 where said system comprises oocytes incorporating said Na⁺ channel, a lipid bilayer incorporating said Na⁺ channel, a mammalian cell incorporating said Na⁺ channel, a drosophila cell incorporating said Na⁺ channel, a bacterial cell incorporating said Na⁺ channel, membrane preparations of any of the foregoing, or vesicle preparations of any of the foregoing.

109. The method of claim 107 where said ASIC component is ASIC1.

110. The method of claim 109 where said Na+ channel further comprises at least one of the components selected from the group consisting of: an ENaC component, a protease component, a PCK component and a syntaxin component.

111. The method of claim 107 where said measuring is accomplished by means of a detecting a label directly or indirectly associated with said test compound.

112. The method of claim 111 where said label is selected from a group consisting of a radiolabel, a fluorescent label, a luminescent label and an enzymatic label.

113. The method of claim 107 where said measuring is accomplished by competition with a labeled competitor and detecting said labeled competitor.

114. A method of identifying agents that modulate a constitutive inward Na⁺ current, said method comprising the steps of:
a. providing a system comprising a Na⁺ channel comprising at least one ASIC component mediating said constitutive inward Na⁺ current in a functional state;
b. contacting said system with a test compound; and
c. measuring said constitutive inward Na⁺ current.

115. The method of claim 114 where said system comprises an oocytes incorporating said Na⁺ channel, a lipid bilayer incorporating said Na⁺ channel, a mammalian cell incorporating said Na⁺ channel, a drosophila cell incorporating said Na⁺ channel, a bacterial cell incorporating said Na⁺ channel, membrane preparations of any of the foregoing, or vesicle preparations of any of the foregoing.

116. The method of claim 114 where said ASIC component is ASIC1.

117. The method of claim 116 where said Na⁺ channel further comprises at least one of the components selected from the group consisting of: an ENaC component, a protease component, a PCK component and a syntaxin component.

118. The method of claim 114 where said modulation is an inhibition of said constitutive inward Na⁺ current.

119. The method of claim 118 where said inhibition is a direct inhibition.

120. The method of claim 118 where said inhibition is an indirect inhibition.

121. The method of claim 114 where said modulation is stimulation of said constitutive inward Na⁺ current.

122. A method of visualizing a tumor in a subject in need of such visualization, said tumor characterized by an expression of a Na⁺ channel mediating a constitutive inward Na⁺ current, said method comprising administering an effective amount of a pharmaceutical composition comprising PcTX1 or a variant of PcTX1 linked to a visualization agent.

123. The method of claim 122 where said visualization agent can be detected during a surgical procedure.

124. The method of claim 123 where said detection aids a healthcare provider in removing said tumor.

125. The method of claim 122 wherein the visualization agent is selected from the group consisting of a radiolabel, a fluorescent label and a luminescent agent.

126. The method of claim 125 wherein the radiolabel is selected from the group consisting of ¹³¹I and ¹²⁵I.

127. The method of claim 122 wherein the tumor is derived from a glial cell.

128. The method of claim 127 wherein the tumor is a glioma.

129. The method of claim 128 wherein the glioma is selected from the group consisting of astrocytoma, glioblastoma and medulloblastoma.

130. The method of claim 122 where the tumor is derived from an epithelial cell.

131. The method of claim 130 where the tumor is a breast carcinoma.

132. The method of claim 122 where the tumor is derived from a melanocyte.

133. The method of claim 133 where the tumor is a melanoma.

134. The method of claim 122 where said Na⁺ channel has an ASIC1 component.

135. The method of claim 134 where said Na⁺ channel further comprises at least one of the components selected from the group consisting of: an ENaC component, a protease component, a PCK component and a syntaxin component.

136. The method of claim 1 where the subject is a human.


SEQUENCE LISTING (5 sequences)

SEQ ID NO: 1 - protein, 40 aa, Psalmopoeus cambridgei
EDCIPKWKGCVNRHGDCCEGLECWKRRRSFEVCVPKTPKT

SEQ ID NO: 2 - protein, 41 aa, Psalmopoeus cambridgei
EDCIPKWKGCVNRHGDCCEGLECWKRRRSFEVCVPKTPKTY

SEQ ID NO: 3 - protein, 40 aa, artificial sequence
  control peptide
VLEPCKWGFRTCKGEDINCPKVWTRERSKCDCEVKRCHGP

SEQ ID NO: 4 - DNA, 20 bp, artificial sequence
  primer
cacaaggagtcggctgtgac

SEQ ID NO: 5 - DNA, 20 bp, artificial sequence
  primer
ggagggtaggtgccacacaa